(19) United States
(12) Patent Application Publication
(10) Pub. No.: US 2010/0298306 A1
(43) Pub. Date: Nov. 25, 2010

(54) (1,4-Diaza-bicyclo[3.2.2]non-6-en-4-yl)-heterocyclyl-methanone Ligands for Nicotinic Acetylcholine Receptors, Useful for the Treatment of Disease

(75) Inventors: Brian Herbert, Stockholm, NJ (US); Richard Schumacher, Monroe, NY (US); Guangxiu Dai, Baton Rouge, LA (US); Wenge Xie, Mahwah, NJ (US)

Correspondence Address: MILLEN, WHITE, ZELANO & BRANIGAN, P.C., 2200 CLARENDON BLVD., SUITE 1400, ARLINGTON, VA, 22201, US

(73) Assignee: MEMORY PHARMACEUTICALS CORPORATION, Montvale, NJ (US)

(21) Appl. No.: 12/739,040

(22) PCT Filed: Oct. 22, 2008
(86) PCT No.: PCT/US2008/080743

§ 371 Date: Aug. 17, 2010

Related U.S. Application Data
(60) Provisional application No. 60/981,643, filed on Oct. 22, 2007.
(60) Provisional application No. 61/050,366, filed on May 5, 2008.

(51) Int. Cl. A61K 31/551; C07D 471/08; A61P 29/00; A61P 25/30; A61P 3/04; A61P 3/10; A61P 25/32; A61P 35/00; A61P 25/28; A61P 25/18; A61P 25/22; A61P 25/24; A61P 25/16; A61P 25/00

(52) U.S. Cl. 514/221; 540/556

(57) ABSTRACT

The present invention relates generally to the field of ligands for nicotinic acetylcholine receptors (nACh receptors), activation of nACh receptors, and the treatment of disease conditions associated with defective or malfunctioning nicotinic acetylcholine receptors, especially of the brain. Further, this invention relates to novel compounds, which act as ligands for the α7 nACh receptor subtype, methods of preparing such compounds, compositions containing such compounds, and methods of use thereof. The novel compounds include compounds of formula I:

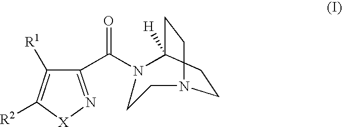

wherein X, R¹, and R² are as herein defined.

This application claims the benefit of U.S. Provisional Application Ser. No. 60/981,643, filed Oct. 22, 2007, and the benefit of U.S. Provisional Application Ser. No. 61/050,366, the entire disclosures of which are hereby incorporated by reference.

### FIELD OF THE INVENTION

The present invention relates generally to the field of ligands for nicotinic acetylcholine receptors (nAChR), activation of nAChRs, and the treatment of disease conditions associated with defective or malfunctioning nicotinic acetylcholine receptors, especially of the brain. Further, this invention relates to novel compounds, which act as ligands for the α7 nAChR subtype, methods of preparing such compounds, compositions comprising such compounds, and methods of use.

### BACKGROUND

There are two types of receptors for the neurotransmitter, acetylcholine: muscarinic receptors and nicotinic receptors, based on the selectivity of action of muscarine and nicotine, respectively. Muscarinic receptors are G-protein coupled receptors. Nicotinic receptors are members of the ligand-gated ion channel family. When activated, the conductance of ions across the nicotinic ion channels increases.

Nicotinic alpha-7 receptor protein forms a homo-pentameric channel in vitro that is highly permeable to a variety of cations (e.g., Ca⁺⁺). Each nicotinic alpha-7 receptor has four transmembrane domains, named M1, M2, M3, and M4. The M2 domain has been suggested to form the wall lining the channel. Sequence alignment shows that nicotinic alpha-7 is highly conserved during evoluion. The M2 domain that lines the channel is identical in protein sequence from chicken to human. For discussions of the alpha-7 receptor, see, e.g., Revah et al. (1991), Nature, 353, 846-849; Galzi et al. (1992), Nature 359, 500-505; Fucile et al. (2000), PNAS 97(7), 3643-3648; Briggs et al. (1999), Eur. J. Pharmacol. 366 (2-3), 301-308; and Gopalakrishnan et al. (1995), Eur. J. Pharmacol. 290(3), 237-246.

The nicotinic alpha-7 receptor channel is expressed in various brain regions and is believed to be involved in many important biological processes in the central nervous system (CNS), including learning and memory. Nicotinic alpha-7 receptors are localized on both presynaptic and postsynaptic terminals and have been suggested to be involved in modulating synaptic transmission. It is therefore of interest to develop novel compounds, which act as ligands for the α7 nAChR subtype, for the treatment of disease conditions associated with defective or malfunctioning nicotinic acetylcholine receptors.

### SUMMARY OF THE INVENTION

This invention relates to novel compounds, which act as ligands for the α7 nAChR subtype, methods of preparing such compounds, compositions comprising such compounds, and methods of use thereof.

### DETAILED DESCRIPTION OF THE INVENTION

The invention includes novel compounds of Formula (I):

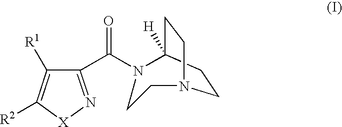

wherein
X is NH, N(CH₃), S or O;
R¹ and R² are each, independently, hydrogen, C₁-C₆-alkyl which is unsubstituted or substituted one or more times by R¹⁰, C₂-C₆-alkenyl which is unsubstituted or substituted one or more times by R¹⁰, C₂-C₆-alkynyl which is unsubstituted or substituted one or more times by R¹⁰, C₃-C₆-cycloalkyl which is unsubstituted or substituted one or more times by R¹¹, C₃-C₈-cycloalkenyl which is unsubstituted or substituted one or more times by R¹¹, halo, OR³, SR³, NR³R⁴, aryl which is unsubstituted or substituted one or more times by R¹², heterocyclyl which is unsubstituted or substituted one or more times by R¹², S(O)ₚR¹³, S(O)ₚNR³R⁴, —C(O)R³, —C(O)OR³, —C(O)NR³R⁴, NO₂, or CN, or
R¹ and R² taken together are

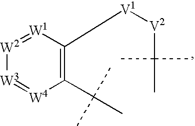

—(CH₂)₂CR⁹═CR⁹—, or —(CH₂)ₘ;
R³ and R⁴ are each, independently, hydrogen, C₁-C₆-alkyl which is unsubstituted or substituted one or more times by R¹⁰, C₃-C₆-alkenyl which is unsubstituted or substituted one or more times by R¹⁰, C₃-C₆-alkynyl which is unsubstituted or substituted one or more times by R¹⁰, C₃-C₆-cycloalkyl which is unsubstituted or substituted one or more times by R¹¹, C₃-C₆-cycloalkenyl which is unsubstituted or substituted one or more times by R¹¹, aryl which is unsubstituted or substituted one or more times by R¹², heterocyclyl which is unsubstituted or substituted one or more times by R¹², —C(O)R⁵, —C(O)OR⁵, or —C(O)NR⁵R⁶;
W¹, W², W³ and W⁴ are each, independently, CR⁷ or N, wherein no more than one of W¹, W², W³ and W⁴ is N;
V¹ and V² are each, independently, O, CR⁸R⁸, S, NH, or NR³, provided that when one of V¹ or V² represent O, S, NH, or NR³, the other is CR⁸R⁸;
m is 3, 4, 5, or 6;
n is 0, 1 or 2;
p is 1 or 2;
R⁵ and R⁶ are each, independently, hydrogen, C₁-C₆-alkyl, C₃-C₆-alkenyl, C₃-C₆-alkynyl, C₃-C₆-cycloalkyl, C₃-C₈-cycloalkenyl, aryl, or heterocyclyl;
R⁷ is hydrogen, C₁-C₆-alkyl which is unsubstituted or substituted one or more times by R¹⁰, C₂-C₆-alkenyl which is unsubstituted or substituted one or more times by R¹⁰, C₂-C₆-alkynyl which is unsubstituted or substituted one or more times by R¹⁰, C₃-C₆-cycloalkyl which is unsubstituted or substituted one or more times by R¹¹, C₃-C₆-cycloalkenyl which is unsubstituted or substituted one or more times by R¹¹, halo, OR³, SR³, NR³R⁴, aryl which is unsubstituted or substituted one or more times by R¹², heterocyclyl which is unsubstituted or substituted one or more times by R¹², S(O)ₚR¹³, S(O)ₚNR³R⁴, —C(O)R³, —C(O)OR³R⁴, —NO₂, or CN;
R⁸ is, in each case independently,
   H,
   C₁-C₆-alkyl which is unsubstituted or substituted one or more times by C₁-C₆-alkoxy, C₁-C₆-haloalkoxy, OH, halo or NR³R⁴,
   O—C₁-C₆-alkyl,
   OH,
   halo, or
   NR³R⁴,
   or two R^{B} together may represent oxo;
   R⁹ is, in each case independently, hydrogen, C₁-C₆-alkyl which is unsubstituted or substituted one or more times by R¹⁰, C₂-C₆-alkenyl which is unsubstituted or substituted one or more times by R¹⁰, C₂-C₆-alkynyl which is unsubstituted or substituted one or more times by R¹⁰, C₃-C₆-cycloalkyl which is unsubstituted or substituted one or more times by R¹¹, C₃-C₈-cycloalkeny which is unsubstituted or substituted one or more times by R¹¹, aryl which is unsubstituted or substituted one or more times by R¹², or heterocyclyl which is unsubstituted or substituted one or more times by R¹²;
   R¹⁰ is, in each case independently, halogen, C₂-C₇-alkoxycarbonyl, hydroxy, C₁-C₆-alkoxy, C₁-C₆-haloalkoxy, C₃-C₆-alkenyloxy, C₃-C₆-alkynyloxy, nitro, amino, C₁₋₆-alkylamino, dialkylamino wherein each alkyl group has independently 1 to 6 carbon atoms, aminocarbonyl, C₁₋₆-alkyl-aminocarbonyl, dialkylaminocarbonyl wherein each alkyl group has independently 1 to 6 carbon atoms, hydroxyalkyl having 1 to 6 carbon atoms, hydroxyalkoxy having 1 to 6 carbon atoms, carboxy, cyano, formyl, alkanoyl having 2 to 7 carbon atoms, benzoyl, C₁-C₆-alkylthio, C₁-C₆-alkylsulfinyl, C₁-C₆-alkylsulfonyl, C₁-C₆-alkylsulfamoyl, phenoxy, formyloxy, alkanoyloxy having 2 to 7 carbon atoms, and benzoyloxy;
   R¹¹ is, in each case independently, halogen, C₁-C₆-alkyl, halogenated C₁-C₆-alkyl, C₃-C₆-alkenyl, C₃-C₆-alkynyl, C₂-C₇-alkoxycarbonyl, hydroxy, C₁-C₆-alkoxy, C₁-C₆-haloalkoxy, C₃-C₆-alkenyloxy, C₃-C₆-alkynyloxy, nitro, methylenedioxy, ethylenedioxy, amino, C₁₋₆-alkylamino, dialkylamino wherein each alkyl group has independently 1 to 6 carbon atoms, aminocarbonyl, C₁₋₆-alkyl-aminocarbonyl, dialkylaminocarbonyl wherein each alkyl group has independently 1 to 6 carbon atoms, hydroxyalkyl having 1 to 6 carbon atoms, hydroxyalkoxy having 1 to 6 carbon atoms, carboxy, cyano, formyl, alkanoyl having 2 to 7 carbon atoms, benzoyl, C₁-C₆-alkylthio, C₁-C₆-alkylsulfinyl, C₁-C₆-alkylsulfonyl, C₁-C₆-alkylsulfamoyl, phenoxy, formyloxy, alkanoyloxy having 2 to 7 carbon atoms, and benzoyloxy;
   R¹² is, in each case independently, halogen, C₁-C₆-alkyl, halogenated C₁-C₆-alkyl, C₃-C₆-alkenyl, C₃-C₆-alkynyl, C₃-C₈-cycloalkyl, C₅-C₈-cycloalkenyl, C₂-C₇-alkoxycarbonyl, hydroxy, C₁-C₆-alkoxy, C₁-C₆-haloalkoxy, C₃-C₆-alkenyloxy, C₃-C₆-alkynyloxy, nitro, methylenedioxy, ethylenedioxy, amino, C₁₋₆-alkylamino, dialkylamino wherein each alkyl group has independently 1 to 6 carbon atoms, aminocarbonyl, C₁₋₆-alkyl-aminocarbonyl, dialkylaminocarbonyl wherein each alkyl group has independently 1 to 6 carbon atoms, hydroxyalkyl having 1 to 6 carbon atoms, hydroxyalkoxy having 1 to 6 carbon atoms, carboxy, cyano, formyl, alkanoyl having 2 to 7 carbon atoms, benzoyl, C₁-C₆-alkylthio, C₁-C₆-alkylsulfinyl, C₁-C₆-alkylsulfonyl, C₁-C₆-alkylsulfamoyl, phenoxy, formyloxy, alkanoyloxy having 2 to 7 carbon atoms, and benzoyloxy; and
   R¹³ is in each case independently, C₁-C₆-alkyl which is unsubstituted or substituted one or more times by R¹⁰, C₃-C₆-alkenyl which is unsubstituted or substituted one or more times by R¹⁰, C₃-C₆-alkynyl which is unsubstituted or substituted one or more times by R¹⁰, C₃-C₆-cycloalkyl which is unsubstituted or substituted one or more times by R¹¹, C₃-C₈-cycloalkenyl which is unsubstituted or substituted one or more times by R¹¹, aryl which is unsubstituted or substituted one or more times by R¹², heterocyclyl which is unsubstituted or substituted one or more times by R¹², —C(O)R⁵, —C(O)OR⁵, or —C(O)NR⁵R⁶;
      and tautomers thereof, pharmaceutically acceptable salts and esters thereof, and wherein if the compound exhibits chirality it can be in the form of a mixture of enantiomers such as a racemate or a mixture of diastereomers, or can be in the form of a single enantiomer or a single diastereomer.

Another embodiment of the invention includes novel compounds of Formula (1) wherein:
R¹ and R² are each, independently, hydrogen, C₁-C₆-alkyl which is unsubstituted or substituted one or more times by R¹⁰, C₂-C₆-alkenyl which is unsubstituted or substituted one or more times by R¹⁰, C₂-C₆-alkynyl which is unsubstituted or substituted one or more times by R¹⁰, C₃-C₆-cycloalkyl which is unsubstituted or substituted one or more times by R¹⁰, C₃-C₈-cycloalkenyl which is unsubstituted or substituted one or more times by R¹⁰, halo, OR³, SR³, NR³R⁴, aryl which is unsubstituted or substituted one or more times by R¹¹, heterocyclyl which is unsubstituted or substituted one or more times by R¹¹, S(O)ₚR¹³, S(O)ₚNR³R⁴, —C(O)R³, —C(O)OR³, —C(O)NR³R⁴, NO₂, or CN, or
R¹ and R² taken together, are

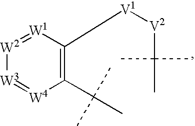

or —(CH₂)ₘ—.

Another embodiment of the invention includes novel compounds of Formula (I)

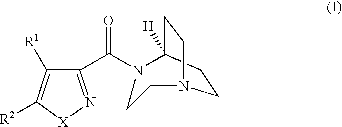

wherein
X is NH, N(CH₃), S or O;
R¹ and R² are each, independently, hydrogen, C₁-C₆-alkyl which is unsubstituted or substituted one or more times by R¹⁰, C₃-C₆-cycloalkyl which is unsubstituted or substituted one or more times by R¹⁰, halo, NH₂, phenyl which is unsubstituted or substituted one or more times by R¹¹, naphthyl which is unsubstituted or substituted one or more times by R¹¹, 1,4-benzodioxan-6-yl which is unsubstituted or substituted one or more times by R¹¹, pyridyl which is unsubstituted or substituted one or more times by R¹¹, thienyl which is unsubstituted or substituted one or more times by R¹¹, or NO₂, or
R¹ and R² taken together, are

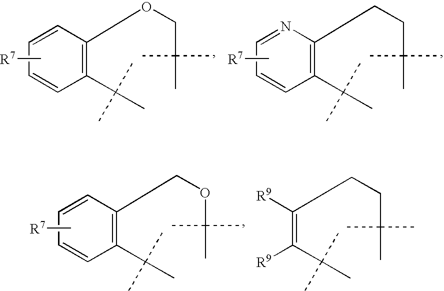

or, —(CH₂)ₘ; and
m is 3, 4, 5, or 6;
   and tautomers thereof, pharmaceutically acceptable salts and esters thereof, and wherein if the compound exhibits chirality it can be in the form of a mixture of enantiomers such as a racemate or a mixture of diastereomers, or can be in the form of a single enantiomer or a single diastereomer.

Another embodiment of the invention includes novel compounds of

Formula (1)
R¹ and R² are each, independently, hydrogen, C₁-C₆-alkyl which is unsubstituted or substituted one or more times by R¹⁰, C₃-C₆-cycloalkyl which is unsubstituted or substituted one or more times by R¹⁰, halo, NH₂, phenyl which is unsubstituted or substituted one or more times by R¹¹, naphthyl which is unsubstituted or substituted one or more times by R¹¹, 1,4-benzodioxan-6-yl which is unsubstituted or substituted one or more times by R¹¹, pyridyl which is unsubstituted or substituted one or more times by R¹¹, thienyl which is unsubstituted or substituted one or more times by R¹¹, or NO₂, or
R¹ and R² taken together, are

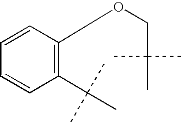

or, —(CH₂)ₘ.

Radicals which are substituted one or more times preferably have 1 to 3 substituents, especially 1 or 2 substituents of the exemplified substituents. Halogenated radicals such as halogenated alkyls are preferably fluorinated and include perhalo radicals such as trifluoromethyl.

The terms identified above have the following meanings throughout: Alkyl throughout means a straight-chain or branched-chain aliphatic hydrocarbon radical having preferably 1 to 6 carbon atoms, particularly 1 to 4 carbon atoms, unless otherwise indicated. Suitable alkyl groups include methyl, ethyl, propyl, isopropyl, butyl, sec-butyl, and tert-butyl. Additional suitable alkyl groups include pentyl, hexyl, 1-, 2- or 3-methylbutyl, 1,1-, 1,2- or 2,2-dimethylpropyl, 1-ethylpropyl, 1-, 2-, 3- or 4-methylpentyl, 1,1-, 1,2-, 1,3-, 2,2-, 2,3- or 3,3-dimethylbutyl, 1- or 2-ethylbutyl, ethylmethylpropyl, trimethylpropyl, and the like. A halogenated alkyl group is an alkyl group which is substituted one or more times by halo (F, Cl, Br, or I). For example, the halogenated alkyl group may be an alkyl group which is substituted one or more times by F (e.g., CF₃, and CHF₂).

When an alkyl group is “substituted,” unless indicated otherwise, it is substituted (i.e., a hydrogen atom may be replaced by a substituent group) one or more times by R¹⁰ groups, i.e., halogen, C₂-C₇-alkoxycarbonyl, hydroxy, C₁-C₆-alkoxy, C₁-C₆-haloalkoxy, C₃-C₆-alkenyloxy, C₃-C₆-alkynyloxy, nitro, amino, C₁₋₆-alkylamino, dialkylamino wherein each alkyl group has independently 1 to 6 carbon atoms, aminocarbonyl, C₁₋₆-alkyl-aminocarbonyl, dialkylaminocarbonyl wherein each alkyl group has independently 1 to 6 carbon atoms, hydroxyalkyl having 1 to 6 carbon atoms, hydroxyalkoxy having 1 to 6 carbon atoms, carboxy, cyano, formyl, alkanoyl having 2 to 7 carbon atoms, benzoyl, C₁-C₆-alkylthio, C₁-C₆-alkylsulfinyl, C₁-C₆-alkylsulfonyl, C₁-C₆-alkylsulfamoyl, phenoxy, formyloxy, alkanoyloxy having 2 to 7 carbon atoms (e.g., acetoxy), and benzoyloxy. For example, where indicated alkyl groups can be substituted by halogen, hydroxy, C₁-C₆-alkoxy, C₁-C₆-haloalkoxy, cyano, nitro, amino, C₁-C₆-alkylamino, and di-C₁-C₆-alkylamino.

Alkenyl throughout means a straight-chain or branched-chain alkyl radical having preferably 2 to 6 carbon atoms, unless otherwise indicated, wherein at least one CH₂CH₂ group is replaced by CH=CH. Suitable alkenyl groups include ethenyl, 1-propenyl, 2-propenyl, 1-butenyl, 2-butenyl, 3-butenyl, 1,3-butadienyl, and 3-methyl-2-butenyl, etc.

When an alkenyl group is “substituted,” unless indicated otherwise, it is substituted (i.e., a hydrogen atom may be replaced by a substituent group) one or more times by R¹⁰ groups, i.e., halogen, C₂-C₇alkoxycarbonyl, hydroxy, C₁-C₆-alkoxy, C₁-C₆-haloalkoxy, C₃-C₆-alkenyloxy, C₃-C₆-alkynyloxy, nitro, amino, C₁₋₆-alkylamino, dialkylamino wherein each alkyl group has independently 1 to 6 carbon atoms, aminocarbonyl, C₁₋₆-alkyl-aminocarbonyl, dialkylaminocarbonyl wherein each alkyl group has independently 1 to 6 carbon atoms, hydroxyalkyl having 1 to 6 carbon atoms, hydroxyalkoxy having 1 to 6 carbon atoms, carboxy, cyano, formyl, alkanoyl having 2 to 7 carbon atoms, benzoyl, C₁-C₆-alkylthio, C₁-C₆-alkylsulfinyl, C₁-C₆-alkylsulfonyl, C₁-C₆-alkylsulfamoyl, phenoxy, formyloxy, alkanoyloxy having 2 to 7 carbon atoms (e.g., acetoxy), and benzoyloxy. For example, where indicated alkyl groups can be substituted by halogen, hydroxy, C₁-C₆-alkoxy, C₁-C₆-haloalkoxy, cyano, nitro, amino, C₁-C₆-alkylamino, and di-C₁-C₆-alkylamino.

Alkynyl throughout means a straight-chain or branched-chain alkyl radical having preferably 2 to 6 carbon atoms, unless otherwise indicated, wherein at least one CH₂CH₂ group is replaced by C≡C. Suitable alkynyl groups include ethynyl, propynyl, butynyl, etc.

When an alkynyl group is “substituted,” unless indicated otherwise, it is substituted (i.e., a hydrogen atom may be replaced by a substituent group) one or more times by R¹⁰ groups, i.e., halogen, C₂-C₇-alkoxycarbonyl, hydroxy, C₁-C₆-alkoxy, C₁-C₆-haloalkoxy, C₃-C₆-alkenyloxy, C₃-C₆-alkynyloxy, nitro, amino, C₁₋₆-alkylamino, dialkylamino wherein each alkyl group has independently 1 to 6 carbon atoms, aminocarbonyl, C₁₋₆-alkyl-aminocarbonyl, dialkylaminocarbonyl wherein each alkyl group has independently 1 to 6 carbon atoms, hydroxyalkyl having 1 to 6 carbon atoms, hydroxyalkoxy having 1 to 6 carbon atoms, carboxy, cyano, formyl, alkanoyl having 2 to 7 carbon atoms, benzoyl, C₁-C₆-alkylthio, C₁-C₆-alkylsulfinyl, C₁-C₆-alkylsulfonyl, C₁-C₆-alkylsulfamoyl, phenoxy, formyloxy, alkanoyloxy having 2 to 7 carbon atoms (e.g., acetoxy), and benzoyloxy. For example, where indicated alkyl groups can be substituted by halogen, hydroxy, C₁-C₆-alkoxy, C₁-C₆-haloalkoxy, cyano, nitro, amino, C₁-C₆-alkylamino, and di-C₁-C₆-alkylamino.

Alkoxy means alkyl-O— groups in which the alkyl portion has preferably 1 to 6 carbon atoms, especially 1 to 4 carbon atoms, unless otherwise indicated. Suitable alkoxy groups or O—C₁-C₆-alkyl groups include methoxy, ethoxy, propoxy, isopropoxy, isobutoxy, and sec-butoxy. The haloalkoxy group is an alkoxy group which is substituted one or more times by halo (F, Cl, Br, or I). For example, the alkoxy group may be substituted one or more times by F (e.g., OCF₃, and OCHF₂).

Aryl, as a group or substituent per se or as part of a group or substituent, refers to an aromatic carbocyclic radical containing 6 to 12 carbon atoms, unless indicated otherwise. Suitable aryl groups include phenyl, naphthyl and biphenyl. Phenyl is preferred.

When an aryl group is “substituted,” unless indicated otherwise, it is substituted (i.e., a hydrogen atom may be replaced by a substituent group) one or more times by R¹² groups, i.e., halogen, C₁-C₆-alkyl, halogenated C₁-C₆-alkyl, C₁-C₆-alkenyl, C₃-C₆-alkynyl, C₃-C₈-cycloalkyl, C₅-C₈-cycloalkenyl, C₂-C₇-alkoxycarbonyl, hydroxy, C₁-C₆-alkoxy, C₁-C₆-haloalkoxy, C₃-C₆-alkenyloxy, C₃-C₆-alkynyloxy, nitro, methylenedioxy, ethylenedioxy, amino, C₁₋₆-alkylamino, dialkylamino wherein each alkyl group has independently 1 to 6 carbon atoms, aminocarbonyl, C₁₋₆-alkyl-aminocarbonyl, dialkylaminocarbonyl wherein each alkyl group has independently 1 to 6 carbon atoms, hydroxyalkyl having 1 to 6 carbon atoms, hydroxyalkoxy having 1 to 6 carbon atoms, carboxy, cyano, formyl, alkanoyl having 2 to 7 carbon atoms, benzoyl, C₁-C₆-alkylthio, C₁-C₆-alkylsulfinyl, C₁-C₆-alkylsulfonyl, C₁-C₆-alkylsulfamoyl, phenoxy, formyloxy, alkanoyloxy having 2 to 7 carbon atoms (e.g., acetoxy), and benzoyloxy. Preferred substituents for the aryl groups include, for example, halogen, phenyl, C₁-C₆-alkyl, halogenated C₁-C₆-alkyl (e.g., trifluoromethyl), hydroxy, C₁-C₆-alkoxy, C₁-C₆-haloalkoxy, cyano, nitro, amino, C₁-C₆-alkylamino, and di-C₁-C₆-alkylamino.

Cycloalkyl means a cyclic, bicyclic or tricyclic saturated hydrocarbon radical having 3 to 8 carbon atoms, unless otherwise indicated. Suitable cycloalkyl groups include cyclopropyl, cyclobutyl, cyclopentyl, and cyclohexyl. Other suitable cycloalkyl groups include spiropentyl, bicyclo[2.2.1]heptyl, and bicyclo[2.2.2]octyl.

When an cycloalkyl group is “substituted,” unless indicated otherwise, it is substituted (i.e., a hydrogen atom may be replaced by a substituent group) one or more times by R¹¹ groups, i.e., halogen, C₁-C₆-alkyl, halogenated C₁-C₆-alkyl, C₃-C₆-alkenyl, C₃-C₆-alkynyl, C₂-C₇-alkoxycarbonyl, hydroxy, C₁-C₆-alkoxy, C₁-C₆-haloalkoxy, C₃-C₆-alkenyloxy, C₃-C₆-alkynyloxy, nitro, methylenedioxy, ethylenedioxy, amino, C₁-₆-alkylamino, dialkylamino wherein each alkyl group has independently 1 to 6 carbon atoms, aminocarbonyl, C₁₋₆-alkyl-aminocarbonyl, dialkylaminocarbonyl wherein each alkyl group has independently 1 to 6 carbon atoms, hydroxyalkyl having 1 to 6 carbon atoms, hydroxyalkoxy having 1 to 6 carbon atoms, carboxy, cyano, formyl, alkanoyl having 2 to 7 carbon atoms, benzoyl, C₁-C₆-alkylthio, C₁-C₆-alkylsulfinyl, C₁-C₆-alkylsulfonyl, C₁-C₆-alkylsulfamoyl, phenoxy, formyloxy, alkanoyloxy having 2 to 7 carbon atoms (e.g., acetoxy), and benzoyloxy. Preferred substituents for the cycloalkyl groups include, for example, F, Cl, Br, C₁-C₄-alkyl, C₁-C₄-alkoxy, hydroxyl, amino, monoalkylamino having 1 to 4 carbon atoms, and/or dialklyamino in which each alkyl group has 1 to 4 carbon atoms.

Cycloalkenyl throughout means a cyclic, bicyclic or tricyclic saturated hydrocarbon radical having 3 to 8 carbon atoms, unless otherwise indicated, wherein at least one CH₂CH₂ group is replaced by CH═CH. Suitable cycloalkenyl groups include cyclopentenyl, cyclohexenyl, cyclooctadienyl, etc.

When an cycloalkenyl group is “substituted,” unless indicated otherwise, it is substituted (i.e., a hydrogen atom may be replaced by a substituent group) one or more times by R¹¹ groups, i.e., halogen, C₁-Cₐ-alkyl, halogenated C₁-C₆-alkyl, C₃-C₆-alkenyl, C₃-C₆-alkynyl, C₂-C₇-alkoxycarbonyl, hydroxy, C₁-C₆-alkoxy, C₁-C₆-haloalkoxy, C₃-C₆-alkenyloxy, C₃-C₆-alkynyloxy, nitro, methylenedioxy, ethylenedioxy, amino, C₁₋₆-alkylamino, dialkylamino wherein each alkyl group has independently 1 to 6 carbon atoms, aminocarbonyl, C₁₋₆-alkyl-aminocarbonyl, dialkylaminocarbonyl wherein each alkyl group has independently 1 to 6 carbon atoms, hydroxyalkyl having 1 to 6 carbon atoms, hydroxyalkoxy having 1 to 6 carbon atoms, carboxy, cyano, formyl, alkanoyl having 2 to 7 carbon atoms, benzoyl, C₁-C₆-alkylthio, C₁-C₆-alkylsulfinyl, C₁-C₆-alkylsulfonyl, C₁-C₆-alkylsulfamoyl, phenoxy, formyloxy, alkanoyloxy having 2 to 7 carbon atoms (e.g., acetoxy), and benzoyloxy. Preferred substituents for the cycloalkenyl groups include, for example, F, Cl, Br, C₁-C₄-alkyl, C₁-C₄-alkoxy, hydroxyl, amino, monoalkylamino having 1 to 4 carbon atoms, and/or dialklyamino in which each alkyl group has 1 to 4 carbon atoms.

Halo means F, Cl, Br, or I.

Heterocyclyl groups refer to saturated, partially saturated and fully unsaturated (i.e., heteroaryl) heterocyclic ring radicals having one, two or three rings, preferably 1 to 2 rings, and a total number of 5 to 14 ring atoms, preferably 5 to 10 ring atoms, wherein at least one of the ring atoms is an N, O or S atom. Preferably, the heterocyclyl group contains 1 to 4 hetero-ring atoms selected from N, O and S, for example, 1 or 2 heteroatoms. Suitable saturated and partially saturated heterocyclyl groups include, but are not limited to tetrahydrofuranyl, tetrahydrothienyl, tetrahydropyranyl, dihydropyranyl, pyrrolidinyl, piperidinyl, piperazinyl, morpholinyl, oxoazolinyl, isoxazolinyl and the like. Suitable heteroaryl groups include but are not limited to furyl, thienyl, pyrrolyl, pyrazolyl, imidazolyl, pyridyl, pyrimidinyl, benzopyranyl, indolyl, quinolinyl, isoquinolinyl, naphthyridinyl and the like. Other examples of suitable heterocyclyl groups, are 2-quinolinyl, 1,3-benzodioxyl, 2-thienyl, 2-benzofuranyl, 2-benzothiophenyl, 3-thienyl, 2,3-dihydro-5-benzofuranyl, 4-indoyl, 4-pyridyl, 3-quinolinyl, 4-quinolinyl, 1,4-benzodioxan-6-yl, 3-indoyl, 2-pyrrolyl, benzopyran-6-yl, 5-indolyl, 1,5-benzoxepin-8-yl, 3-pyridyl, 6-coumarinyl, 5-benzofuranyl, 2-isoimidazol-4-yl, 3-pyrazolyl, 3-carbazolyl, 2-thiazolyl, 2-oxazolyl, 1-imidazolyl, and 2-imidazolyl.

When a heterocyclyl group is characterized as “optionally substituted”, it can be substituted (i.e., a hydrogen atom may be replaced by a substituent group) one or more times by suitable substituents including halogen, C₁-C₆-alkyl, halogenated C₁-C₆-alkyl, C₃-C₆-alkenyl, C₃-C₈-alkynyl, C₃-C₈-cycloalkyl, C₅-C₈-cycloalkenyl, C₂-C₇-alkoxycarbonyl, hydroxy, C₁-C₆-alkoxy, C₁-C₆-haloalkoxy, C₃-C₆-alkenyloxy, C₃-C₆-alkynyloxy, nitro, methylenedioxy, ethylenedioxy, amino, C dialkylamino wherein each alkyl group has independently 1 to 6 carbon atoms, aminocarbonyl, C₁₋₆-alkyl-aminocarbonyl, dialkylaminocarbonyl wherein each alkyl group has independently 1 to 6 carbon atoms, hydroxyalkyl having 1 to 6 carbon atoms, hydroxyalkoxy having 1 to 6 carbon atoms, carboxy, cyano, formyl, alkanoyl having 2 to 7 carbon atoms, benzoyl, C₁-C₆-alkylthio, C₁-C₆-alkylsulfinyl, C₁-C₆-alkylsulfonyl, C₁-C₆-alkylsulfamoyl, aryl having 6 to 12 carbon atoms, phenoxy, formyloxy, alkanoyloxy having 2 to 7 carbon atoms (e.g., acetoxy), and benzoyloxy. Preferred substituents for the heterocyclyl groups include, for example, halogen, phenyl, C₁-C₆-alkyl, halogenated C₁-C₆-alkyl (e.g., trifluoromethyl), hydroxy, C₁-C₆-alkoxy, C₁-C₆-haloalkoxy, cyano, nitro, oxo, amino, C₁-C₆-alkylamino, and di-C₁-C₆-alkylamino.

According to a further aspect of the invention, R¹ and R² are each independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, carbamoyl (NH₂—CO—), phenyl, methylphenyl, ethyiphenyl, methoxyphenyl, ethoxyphenyl, fluorophenyl, chlorophenyl, aminophenyl, cyanophenyl, nitrophenyl, naphthyl, pyridyl, methylpyridyl, ethylpyridyl, methoxypyridyl, ethoxypyridyl, fluoropyridyl, chioropyridyl, aminopyridyl, cyanopyridyl, nitropyridyl, thienyl, methylthienyl, fluorothienyl, chlorothienyl, pyazole, or methylpyrazole.

According to a further aspect of the invention, R¹ is H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—), and/or R² is H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, carbamoyl (NH₂—CO—), phenyl, methylphenyl, ethylphenyl, methoxyphenyl, ethoxyphenyl, fluorophenyl, chlorophenyl, aminophenyl, cyanophenyl, nitrophenyl, naphthyl, pyridyl, methylpyridyl, ethylpyridyl, methoxypyridyl, ethoxypyridyl, fluoropyridyl, chloropyridyl, aminopyridyl, cyanopyridyl, nitropyridyl, thienyl, methyithienyl, fluorothienyl, chlorothienyl, pyazole, or methylpyrazole.

According to a further aspect of the invention, R¹ is H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—), and/or R² is phenyl, methylphenyl, ethylphenyl, methoxyphenyl, ethoxyphenyl, fluorophenyl, chlorophenyl, aminophenyl, cyanophenyl, nitrophenyl, naphthyl, pyridyl, methylpyridyl, ethylpyridyl, methoxypyridyl, ethoxypyridyl, fluoropyridyl, chloropyridyl, aminopyridyl, cyanopyridyl, nitropyridyf, thienyl, methyithienyl, fluorothienyl, chlorothienyl, pyazole, or methylpyrazole.

According to a further aspect of the invention, R¹ and R² together are —(CH₂)₃—, —(CH₂)₄—, —(CH₂)₅—, or —(CH₂)₆—, especially —(CH₂)₃— or —(CH₂)₆—.

According to a further aspect of the invention, R¹ and R² together are

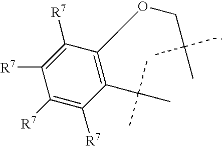

According to a further aspect of the invention, X is preferably NH, S or O, especially O.

According to a further aspect, the invention relates to compounds of Formula Ia:

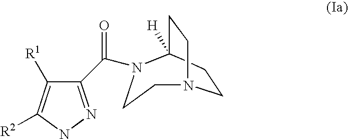

wherein X, R¹, and R² are as defined in Formula I.

According to a further aspect, the invention relates to compounds of Formula la wherein R¹ and R² are each independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, carbamoyl (NH₂—CO—), phenyl, methylphenyl, ethylphenyl, methoxyphenyl, ethoxyphenyl, fluorophenyl, chlorophenyl, aminophenyl, cyanophenyl, nitrophenyl, naphthyl, pyridyl, methylpyridyl, ethylpyridyl, methoxypyridyl, ethoxypyridyl, fluoropyridyl, chloropyridyl, aminopyridyl, cyanopyridyl, nitropyridyl, thienyl, methylthienyl, fluorothienyl, chlorothienyl, pyazole, or methylpyrazole.

According to a further aspect, the invention relates to compounds of Formula la wherein R¹ is H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—), and/or R² is H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, carbamoyl (NH₂—CO—), phenyl, methylphenyl, ethylphenyl, methoxyphenyl, ethoxyphenyl, fluorophenyl, chlorophenyl, aminophenyl, cyanophenyl, nitrophenyl, naphthyl, pyridyl, methylpyridyl, ethylpyridyl, methoxypyridyl, ethoxypyridyl, fluoropyridyl, chloropyridyl, aminopyridyl, cyanopyridyl, nitropyridyl, thienyl, methylthienyl, fluorothienyl, chlorothienyl, pyazole, or methylpyrazole.

According to a further aspect, the invention relates to compounds of Formula Ia wherein R¹ is H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, and carbamoyl (NH₂—CO—), and/or R² is selected from phenyl, methylphenyl, ethylphenyl, methoxyphenyl, ethoxyphenyl, fluorophenyl, chlorophenyl, aminophenyl, cyanophenyl, nitrophenyl, naphthyl, pyridyl, methylpyridyl, ethylpyridyl, methoxypyridyl, ethoxypyridyl, fluoropyridyl, chloropyridyl, aminopyridyl, cyanopyridyl, nitropyridyl, thienyl, methylthienyl, fluorothienyl, chlorothienyl, pyazole, or methylpyrazole.

According to a further aspect, the invention relates to compounds of Formula Ia wherein R¹ and R² together are —(CH₂)₃—, —(CH₂)₄—, —(CH₂)₅—, or —(CH₂)₆—, especially —(CH₂)₃— or —(CH₂)₆—.

According to a further aspect; the invention relates to compounds of Formula Ib:

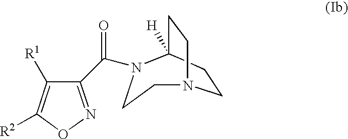

wherein X, R¹, and R² are as defined in Formula I.

According to a further aspect, the invention relates to compounds of Formula Ib wherein R¹ and R² are each independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, carbamoyl (NH₂—CO—), phenyl, methylphenyl, ethylphenyl, methoxyphenyl, ethoxyphenyl, fluorophenyl, chlorophenyl, aminophenyl, cyanophenyl, or nitrophenyl.

According to a further aspect, the invention relates to compounds of Formula Ic:

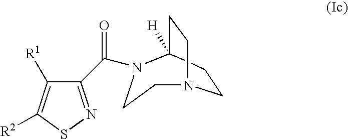

wherein X, R¹, and R² are as defined in Formula I.

According to a further aspect, the invention relates to compounds of Formula Ic wherein R¹ and R² are each independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, carbamoyl (NH₂—CO—), phenyl, methylphenyl, ethylphenyl, methoxyphenyl, ethoxyphenyl, fluorophenyl, chiorophenyl, aminophenyl, cyanophenyl, or nitrophenyl.

According to a further aspect, the invention relates to compounds of Formula Id:

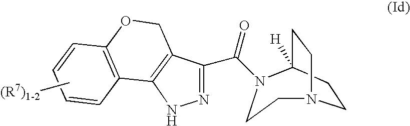

wherein R⁷ is as defined in Formula I.

According to a further aspect, the invention relates to compounds of Formula Id wherein R⁷ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, carbamoyl (NH₂—CO—), or optionally substituted heterocyclyl (e.g., methoxypyrrolidinyl).

According to a further aspect, the invention relates to compounds of Formula Id wherein there is only one R⁷ group and it is H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

According to a further aspect, the invention relates to compounds of Formula Id wherein R⁷ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

According to a further aspect, the invention relates to compounds of Formula Ie:

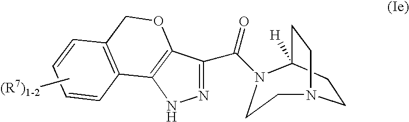

wherein R⁷ is as defined in Formula I.

According to a further aspect, the invention relates to compounds of Formula Ie wherein R⁷ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

According to a further aspect, the invention relates to compounds of Formula Ie wherein R⁷ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

According to a further aspect, the invention relates to compounds of Formula If:

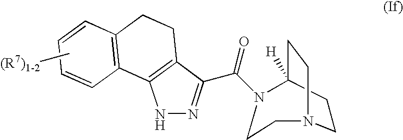

wherein R⁷ is as defined in Formula I.

According to a further aspect, the invention relates to compounds of Formula If wherein R⁷ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

According to a further aspect, the invention relates to compounds of Formula If wherein R⁷ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

According to a further aspect, the invention relates to compounds of Formula Ig:

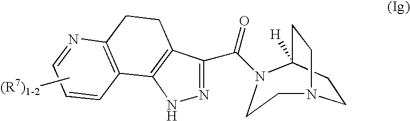

wherein R⁷ is as defined in Formula I.

According to a further aspect, the invention relates to compounds of Formula Ig wherein R⁷ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

According to a further aspect, the invention relates to compounds of Formula Ig wherein R⁷ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

According to a further aspect, the invention relates to compounds of Formula Ih:

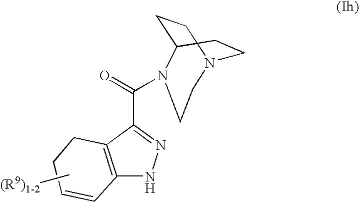

wherein, R⁹ is as defined in Formula I.

According to a further aspect, the invention relates to compounds of Formula Ih wherein R⁹ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

According to a further aspect, the invention relates to compounds of Formula Ih wherein R⁹ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

According to a further aspect, the invention relates to the following compounds of Formula I:
4-[(4-Chloro-1H-pyrazol-3-yl)carbonyl]-1,4-diazabicyclo[3.2.2]nonane,
4-[(4-nitro-1H-pyrazol-3-yl)carbonyl]-1,4-diazabicyclo[3.2.2]nonane,
4-[(5-Methyl-1H-pyrazol-3-yl)carbonyl]-1,4-diazabicyclo[3.2.2]nonane,
4-(1H-pyrazol-3-ylcarbonyl)-1,4-diazabicyclo[3.2.2]nonane,
4-{[5-(4-Methoxyphenyl)-1H-pyrazol-3-yl]carbonyl}-1,4-diazabicyclo[3.2.2]nonane,
4-[(5-Phenylisoxazol-3-yl)carbonyl]-1,4-diazabicyclo[3.2.2]nonane,
4-{[5-(2-thienyl)-1H-pyrazol-3-yl]carbonyl}-1,4-diazabicyclo[3.2.2]nonane,
4-[(5-Phenyl-1H-pyrazol-3-yl)carbonyl]-1,4-diazabicyclo[3.2.2]nonane,
4-[(5-Cyclopropyl-1H-pyrazol-3-yl)carbonyl]-1,4-diazabicyclo[3.2.2]nonane,
4-{[5-(3-Methoxyphenyl)-1H-pyrazol-3-yl]carbonyl}-1,4-diazabicyclo[3.2.2]nonane,
4-(1,4,5,6-Tetrahydrocyclopenta[c]pyrazol-3-ylcarbonyl)-1,4-diazabicyclo[3.2.2]nonane,
4-{[5-(2-Methoxyphenyl)-1H-pyrazol-3-yl]carbonyl}-1,4-diazabicyclo[3.2.2]nonane,
4-[(4-Bromo-1H-pyrazol-3-yl)carbonyl]-1,4-diazabicyclo[3.2.2]nonane,
3-(1,4-Diazabicyclo[3.2.2]non-4-ylcarbonyl)isothiazol-4-amine,
3-(1,4-Diazabicyclo[3.2.2]non-4-ylcarbonyl)-1,4-dihydrochromeno[4,3-c]pyrazole,
3-(1,4-Diazabicyclo[3.2.2]non-4-ylcarbonyl)-4,5,6,7,8,9-hexahydro-1H-cycloocta[c]pyrazole,
4-{[5-(2,3-Dihydro-1,4-benzodioxin-6-yl)-1H-pyrazol-3-yl]carbonyl}-1,4-diazabicyclo[3.2.2]nonane,
4-{[5-(2-Naphthyl)-1H-pyrazol-3-yl]carbonyl}-1,4-diazabicyclo[3.2.2]nonane,
4-{[5-(3-Thienyl)-1H-pyrazol-3-yl]carbonyl}-1,4-diazabicyclo[3.2.2]nonane,
4-{[5-(4-Fluorophenyl)-1H-pyrazol-3-yl]carbonyl}-1,4-diazabicyclo[3.2.2]nonane,
4-[(5-Pyridin-2-yl-1H-pyrazol-3-yl)carbonyl]-1,4-diazabicyclo[3.2.2]nonane,
4-[(5-Pyridin-4-yl-1H-pyrazol-3-yl)carbonyl]-1,4-diazabicyclo[3.2.2]nonane,
4-[(5-Pyridin-3-yl-1H-pyrazol-3-yl)carbonyl]-1,4-diazabicyclo[3.2.2]nonane,
5′-(1,4-Diazabicyclo[3.2.2]non-4-ylcarbonyl)-1-methyl-1H,2′H-3,3′-bipyrazole,
3-(1,4-Diazabicyclo[3.2.2]non-4-ylcarbonyl)-6-methoxy-1,4-dihydrochromeno[4,3-c]pyrazole,
3-(1,4-Diazabicyclo[3.2.2]non-4-ylcarbonyl)-8-methoxy-1,4-dihydrochromeno[4,3-c]pyrazole,
3-(1,4-Diazabicyclo[3.2.2]non-4-ylcarbonyl)-7-methoxy-1,4-dihydrochromeno[4,3-c]pyrazole,
3-(1,4-Diazabicyclo[3.2.2]non-4-ylcarbonyl)-6-(4-fluorophenyl)-4,5-dihydro-1H-indazole
4-(Isothiazol-3-ylcarbonyl)-1,4-diazabicyclo[3.2.2]nonane
4-[(5-Bromoisothiazol-3-yl)carbonyl]-1,4-diazabicyclo[3.2.2]nonane
3-(1,4-Diazabicyclo[3.2.2]non-4-ylcarbonyl)-4,5-dihydro-1H-benzo[g]indazole
3-(1,4-Diazabicyclo[3.2.2]non-4-ylcarbonyl)-1,5-dihydroisochromeno[4,3-c]pyrazole
3-(1,4-Diazabicyclo[3.2.2]non-4-ylcarbonyl)-4,5-dihydro-1H-pyrazolo[3,4-f]quinoline
3-(1,4-Diazabicyclo[3.2.2]non-4-ylcarbonyl)-7-[(3S)-3-methoxypyrrolidin-1-yl]-1,4-dihydrochromeno[4,3-c]pyrazole
7-Bromo-3-(1,4-diazabicyclo[3.2.2]non-4-ylcarbonyl)-1,4-dihydrochromeno[4,3-c]pyrazole,
3-(1,4-Dzabicyclo[3.2.2]non-4-ylcarbonyl)-1H-pyrazol-5-amine,
   and tautomers thereof, and pharmaceutically acceptable salts thereof (such as a hydrochloride salt), and

wherein if the compound exhibits chirality it can be in the form of a mixture of enantiomers such as a racemate or a mixture of diastereomers, or can be in the form of a single enantiomer or a single diastereomer.

According to a further aspect, the invention relates to the following compounds of Formula I:
4-[(4-Chloro-1H-pyrazol-3-yl)carbonyl]-1,4-diazabicyclo[3.2.2]nonane,
4-[(4-nitro-1H-pyrazol-3-yl)carbonyl]-1,4-diazabicyclo[3.2.2]nonane,
4-[(5-Methyl-1H-pyrazol-3-yl)carbonyl]-1,4-diazabicyclo[3.2.2]nonane,
4-(1H-pyrazol-3-ylcarbonyl)-1,4-diazabicyclo[3.2.2]nonane,
4-{[5-(4-Methoxyphenyl)-1H-pyrazol-3-yl]carbonyl}-1,4-diazabicyclo[3.2.2]nonane,
4-[(5-Phenylisoxazol-3-yl)carbonyl]-1,4-diazabicyclo[3.2.2]nonane,
4-{[5-(2-thienyl)-1H-pyrazol-3-yl]carbonyl}-1,4-diazabicyclo[3.2.2]nonane,
4-[(5-Phenyl-1H-pyrazol-3-yl)carbonyl]-1,4-diazabicyclo[3.2.2]nonane,
4-[(5-Cyclopropyl-1H-pyrazol-3-yl)carbonyl]-1,4-diazabicyclo[3.2.2]nonane,
4-{[5-(3-Methoxyphenyl)-1H-pyrazol-3-yl]carbonyl}-1,4-diazabicyclo[3.2.2]nonane,
4-(1,4,5,6-Tetrahydrocyclopenta[c]pyrazol-3-ylcarbonyl)-1,4-diazabicyclo[3.2.2]nonane,
4-{[5-(2-Methoxyphenyl)-1H-pyrazol-3-yl]carbonyl}-1,4-diazabicyclo[3.2.2]nonane,
4-[(4-Bromo-1H-pyrazol-3-yl)carbonyl]-1,4-diazabicyclo[3.2.2]nonane,
3-(1,4-Diazabicyclo[3.2.2]non-4-ylcarbonyl)isothiazol-4-amine,
3-(1,4-Diazabicyclo[3.2.2]non-4-ylcarbonyl)-1,4-dihydrochromeno[4,3-c]pyrazole,
3-(1,4-Diazabicyclo[3.2.2]non-4-ylcarbonyl)-4,5,6,7,8,9-hexahydro-1H-cycloocta[c]pyrazole,
4-{[5-(2,3-Dihydro-1,4-benzodioxin-6-yl)-1H-pyrazol-3-yl]carbonyl}-1,4-diazabicyclo[3.2.2]nonane,
4-{[5-(2-Naphthyl)-1H-pyrazol-3-yl]carbonyl}-1,4-diazabicyclo[3.2.2]nonane,
4-{[5-(3-Thienyl)-1H-pyrazol-3-yl]carbonyl}-1,4-diazabicyclo[3.2.2]nonane,
4-{[5-(4-Fluorophenyl)-1H-pyrazol-3-yl]carbonyl}-1,4-diazabicyclo[3.2.2]nonane,
4-[(5-Pyridin-2-yl-1H-pyrazol-3-yl)carbonyl]-1,4-diazabicyclo[3.2.2]nonane,
4-[(5-Pyridin-4-yl-1H-pyrazol-3-yl)carbonyl]-1,4-diazabicyclo[3.2.2]nonane,
4-[(5-Pyridin-3-yl-1H-pyrazol-3-yl)carbonyl]-1,4-diazabicyclo[3.2.2]nonane,
5′-(1,4-Diazabicyclo[3.2.2]non-4-ylcarbonyl)-1-methyl-1H,2′H-3,3′-bipyrazole,
3-(1,4-Diazabicyclo[3.2.2]non-4-ylcarbonyl)-6-methoxy-1,4-dihydrochromeno[4,3-c]pyrazole,
3-(1,4-Diazabicyclo[3.2.2]non-4-ylcarbonyl)-8-methoxy-1,4-dihydrochromeno[4,3-c]pyrazole,
3-(1,4-Diazabicyclo[3.2.2]non-4-ylcarbonyl)-7-methoxy-1,4-dihydrochromeno[4,3-c]pyrazole,
3-(1,4-Diazabicyclo[3.2.2]non-4-ylcarbonyl)-6-(4-fluorophenyl)-4,5-dihydro-1H-indazole
4-(Isothiazol-3-ylcarbonyl)-1,4-diazabicyclo[3.2.2]nonane
4-[(5-Bromoisothiazol-3-yl)carbonyl]-1,4-diazabicyclo[3.2.2]nonane
3-(1,4-Diazabicyclo[3.2.2]non-4-ylcarbonyl)-4,5-dihydro-1H-benzo[g]indazole
3-(1,4-Diazabicyclo[3.2.2]non-4-ylcarbonyl)-1,5-dihydroisochromeno[4,3-c]pyrazole
3-(1,4-Diazabicyclo[3.2.2]non-4-ylcarbonyl)-4,5-dihydro-1H-pyrazolo[3,4-f]quinoline
3-(1,4-Diazabicyclo[3.2.2]non-4-ylcarbonyl)-7-[(3S)-3-methoxypyrrolidin-1-yl]-1,4-dihydrochromeno[4,3-c]pyrazole
7-Bromo-3-(1,4-diazabicyclo[3.2.2]non-4-ylcarbonyl)-1,4-dihydrochromeno[4,3-c]pyrazole,
   and tautomers thereof, and pharmaceutically acceptable salts thereof (such as a hydrochloride salt), and

wherein if the compound exhibits chirality it can be in the form of a mixture of enantiomers such as a racemate or a mixture of diastereomers, or can be in the form of a single enantiomer or a single diastereomer.

According to a further aspect, the invention relates to the following compounds of Formula I:
4-[(4-Chloro-1H-pyrazol-3-yl)carbonyl]-1,4-diazabicyclo[3.2.2]nonane,
4-[(4-nitro-1H-pyrazol-3-yl)carbonyl]-1,4-diazabicyclo[3.2.2]nonane,
4-[(5-Methyl-1H-pyrazol-3-yl)carbonyl]-1,4-diazabicyclo[3.2.2]nonane,
4-(1H-pyrazol-3-ylcarbonyl)-1,4-diazabicyclo[3.2.2]nonane,
4-{[5-(4-Methoxyphenyl)-1H-pyrazol-3-yl]carbonyl}-1,4-diazabicyclo[3.2.2]nonane,
4-[(5-Phenylisoxazol-3-yl)carbonyl]-1,4-diazabicyclo[3.2.2]nonane,
4-{[5-(2-thienyl)-1H-pyrazol-3-yl]carbonyl}-1,4-diazabicyclo[3.2.2]nonane,
4-[(5-Phenyl-1H-pyrazol-3-yl)carbonyl]-1,4-diazabicyclo[3.2.2]nonane,
4-[(5-Cyclopropyl-1H-pyrazol-3-yl)carbonyl]-1,4-diazabicyclo[3.2.2]nonane,
4-{[5-(3-Methoxyphenyl)-1H-pyrazol-3-yl]carbonyl}-1,4-diazabicyclo[3.2.2]nonane,
4-(1,4,5,6-Tetrahydrocyclopenta[c]pyrazol-3-ylcarbonyl)-1,4-diazabicyclo[3.2.2]nonane,
4-{[5-(2-Methoxyphenyl)-1H-pyrazol-3-yl]carbonyl}-1,4-diazabicyclo[3.2.2]nonane,
4-[(4-Bromo-1H-pyrazol-3-yl)carbonyl]-1,4-diazabicyclo[3.2.2]nonane,
3-(1,4-Diazabicyclo[3.2.2]non-4-ylcarbonyl)isothiazol-4-amine,
3-(1,4-Diazabicyclo[3.2.2]non-4-ylcarbonyl)-1,4-dihydrochromeno[4,3-c]pyrazole,
3-(1,4-Diazabicyclo[3.2.2]non-4-ylcarbonyl)-4,5,6,7,8,9-hexahydro-1H-cycloocta[c]pyrazole,
4-{[5-(2,3-Dihydro-1,4-benzodioxin-6-yl)-1H-pyrazol-3-yl]carbonyl}-1,4-diazabicyclo[3.2.2]nonane,
4-{[5-(2-Naphthyl)-1H-pyrazol-3-yl]carbonyl}-1,4-diazabicyclo[3.2.2]nonane,
4-{[5-(3-Thienyl)-1H-pyrazol-3-yl]carbonyl}-1,4-diazabicyclo[3.2.2]nonane,
4-{[5-(4-Fluorophenyl)-1H-pyrazol-3-yl]carbonyl}-1,4-diazabicyclo[3.2.2]nonane,
4-[(5-Pyridin-2-yl-1H-pyrazol-3-yl)carbonyl]-1,4-diazabicyclo[3.2.2]nonane,
4-[(5-Pyridin-4-yl-1H-pyrazol-3-yl)carbonyl]-1,4-diazabicyclo[3.2.2]nonane,
4-[(5-Pyridin-3-yl-1H-pyrazol-3-yl)carbonyl]-1,4-diazabicyclo[3.2.2]nonane,
5′-(1,4-Diazabicyclo[3.2.2]non-4-ylcarbonyl)-1-methyl-1H,2′H-3,3′-bipyrazole,
3-(1,4-Diazabicyclo[3.2.2]non-4-ylcarbonyl)-6-methoxy-1,4-dihydrochromeno[4,3-c]pyrazole,
3-(1,4-Diazabicyclo[3.2.2]non-4-ylcarbonyl)-8-methoxy-1,4-dihydrochromeno[4,3-c]pyrazole,
3-(1,4-Diazabicyclo[3.2.2]non-4-ylcarbonyl)-7-methoxy-1,4-dihydrochromeno[4,3-c]pyrazole,
   and tautomers thereof, and pharmaceutically acceptable salts thereof (such as a hydrochloride salt), and

wherein if the compound exhibits chirality it can be in the form of a mixture of enantiomers such as a racemate or a mixture of diastereomers, or can be in the form of a single enantiomer or a single diastereomer.

According to a further aspect, the invention relates to the following compounds:
(1H-Pyrazol-3-ylcarbonyl)-1,4-diazabicyclo[3.2.2]nonane hydrochloride, and
3-(1,4-Diazabicyclo[3.2.2]non-4-ylcarbonyl)-1,4-dihydrochromeno[4,3-c]pyrazole hydrochloride,

wherein if the compound exhibits chirality it can be in the form of a mixture of enantiomers such as a racemate or a mixture of diastereomers, or can be in the form of a single enantiomer or a single diastereomer.

Alternative Forms of Novel Compounds [63] Also included in the compounds of the present invention are (a) the stereoisomers thereof, (b) the pharmaceutically acceptable salts thereof, (c) the tautomers thereof, (d) the protected acids and the conjugate acids thereof, and (e) the prodrugs thereof.

One of ordinary skill in the art will recognize that compounds of Formula I can exist in different geometrical isomeric forms. Examples of geometric isomers include, but are not limited to, cis isomers or trans isomers across a double bond. In addition, some of the compounds of the present invention possess one or more asymmetric atoms and are thus capable of existing in the form of optical isomers, as well as in the form of racemic or nonracemic mixtures thereof, and in the form of diastereomers and diastereomeric mixtures inter alia. All of these compounds, including cis isomers, trans isomers, diastereomic mixtures, racemates, nonracemic mixtures of enantiomers, substantially pure, and pure enantiomers, are within the scope of the present invention. Substantially pure enantiomers contain no more than 5% w/w of the corresponding opposite enantiomer, preferably no more than 2%, most preferably no more than 1%.

The optical isomers can be obtained by resolution of the racemic mixtures according to conventional processes, for example, by the formation of diastereomeric salts using an optically active acid or base or formation of covalent diastereomers.

Examples of appropriate acids are tartaric, diacetyltartaric, dibenzoyltartaric, ditoluoyltartaric and camphorsulfonic acid. Mixtures of diastereomers can be separated into their individual diastereomers on the basis of their physical and/or chemical differences by methods known to those skilled in the art, for example, by chromatography or fractional crystallization. The optically active bases or acids are then liberated from the separated diastereomeric salts.

A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivitization, are also useful. The optically active compounds of Formula I can likewise be obtained by utilizing optically active starting materials in chiral syntheses processes under reaction conditions which do not cause racemization.

In addition, one of ordinary skill in the art will recognize that the compounds can be used in different enriched isotopic forms, e.g., enriched in the content of ²H, ³, ¹¹C, ¹³C and/or ¹⁴C. In one particular embodiment, the compounds are deuterated. Such deuterated forms can be made by the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] (2000), 110 pp. CAN 133:68895 AN 2000:473538 CAPLUS; Kabalka, George W.; Varma, Rajender S. The Synthesis of Radiolabeled Compounds via Organometallic Intermediates. Tetrahedron (1989), 45(21), 6601-21, CODEN: TETRAB ISSN:0040-4020. CAN 112:20527 AN 1990:20527 CAPLUS; and Evans, E. Anthony. Synthesis of radiolabeled compounds, J. Radioanal. Chem. (1981), 64(1-2), 9-32. CODEN: JRACBN ISSN:0022-4081, CAN 95:76229 AN 1981:476229 CAPLUS.

Pharmaceutically acceptable salts of the compounds of the present invention include salts commonly used to form alkali metal salts or form addition salts of free acids or free bases. The nature of the salt is not critical, provided that it is pharmaceutically-acceptable. Suitable pharmaceutically acceptable acid addition salts may be prepared from an inorganic acid or from an organic acid. Examples of such inorganic acids include, but are not limited to, hydrochloric, hydrobromic, hydroiodic, nitric, carbonic, sulfuric, and phosphoric acid. Appropriate organic acids may be selected from aliphatic, cycloaliphatic, aromatic, heterocyclic, carboxylic, and sulfonic classes of organic acids. Examples of organic and sulfonic classes of organic acids includes, but are not limited to, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, N-hydroxybutyric, salicylic, galactaric, and galacturonic acid, and combinations thereof.

Tautomers of the compounds of the invention are encompassed by the present invention. Thus, for example, a carbonyl includes its hydroxy tautomer. The protected acids include, but are not limited to, esters, hydroxyamino derivatives, amides and sulfonamides.

The present invention includes the prodrugs and salts of the prodrugs.

Formation of prodrugs is well known in the art in order to enhance the properties of the parent compound; such properties include solubility, absorption, biostability, and release time (see, e.g., “Pharmaceutical Dosage Form and Drug Delivery Systems” (Sixth Edition), edited by Ansel et al., publ. by Williams & Wilkins, pgs. 27-29, (1995), which is hereby incorporated by reference). Commonly used prodrugs are designed to take advantage of the major drug biotransformation reactions, and are also to be considered within the scope of the invention. Major drug biotransformation reactions include N-dealkylation, O-dealkylation, aliphatic hydroxylation, aromatic hydroxylation, N-oxidation, S-oxidation, deamination, hydrolysis reactions, glucuronidation, sulfation, and acetylation (see, e.g., Goodman and Gilman's The Pharmacological Basis of Therapeutics (Ninth Edition), editor Molinoff et al., publ. by McGraw-Hill, pages 11-13, (1996), which is hereby incorporated by reference).

Preferred aspects include pharmaceutical compositions comprising a compound of this invention and a pharmaceutically acceptable carrier and, optionally, another active agent as discussed below; a method of stimulating, activating or inhibiting alpha-7 nicotinic receptors, e.g., as determined by a conventional assay or one described herein, either in vitro or in vivo (in an animal, e.g., in an animal model, or in a mammal or in a human); a method of treating a neurological syndrome, e.g., loss of memory, especially long-term memory, cognitive impairment or decline, memory impairment, etc.; and a method of treating a disease state modulated by nicotinic alpha-7 activity, in a mammal, e.g., a human, e.g., those mentioned herein.

### General Preparative Methods

In general, the compounds used in this invention may be prepared by standard techniques known in the art, by known processes analogous thereto, and/or by the processes described herein, using starting materials which are either commercially available or producible according to routine, conventional chemical methods. The following preparative methods are presented to aid the reader in the synthesis of the compounds of the present invention.

Additionally, sensitive or reactive groups on a compound of Formula (I) may need to be protected and deprotected during any of the above methods. Protecting groups in general may be added and removed by conventional methods well known in the art (see, e.g., T. W. Greene and P. G. M. Wuts, Protective Groups in Organic Synthesis; Wiley: New York, (1999)).

The particular process to be utilized in the preparation of the compounds of this invention depends upon the specific compound desired. Such factors as the selection of the specific moiety, and the specific substituents possible at various locations on the molecule, all play a role in the path to be followed in the preparation of the specific compounds of this invention. Those factors are readily recognized by one of ordinary skill in the art.

The compounds of the present invention may be prepared conventionally. Some of the known processes that can be used are described below. All starting materials are known or can be conventionally prepared from known starting materials.

The synthesis of similar compounds is disclosed in copending application Ser. No. 11/123,219, filed May 6, 2005, which claims the benefit of U.S. Provisional Application Ser. No. 60/568,696, filed May 7, 2004, U.S. Provisional Application Ser. No. 60/574,712, filed May 27, 2004, and U.S. Provisional Application Ser. No. 60/626,469, filed Nov. 10, 2004, the entire disclosures of each of which are hereby incorporated by reference.

Acids for use in the preparation of the bicyclobase amides are either commercially available, or prepared by straightforward methods known to those skilled in the art.

The bicycloamine for use in the preparation of the bicyclobase amides is commercially available (Olainfarm). The bicyclobase amide can be prepared by the coupling reaction of acids with the bicycloamine and HATU or HBTU in DMF. The couplings are generally performed at room temperatures for 18-24 hours. The resultant adducts are isolated and purified by standard techniques, such as chromatography or recrystallization, practiced by those skilled in the art.

Compounds of Formula (I) are most generally prepared by the coupling reaction depicted in Reaction Scheme 1.

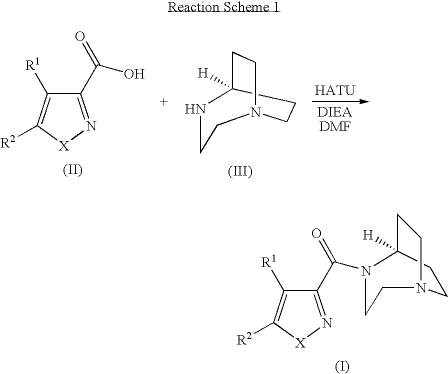

In this scheme, a heterocyclic carboxylic acid of formula (II) is coupled with a bicyclic amine of formula (III), facilitated by a coupling reagent such as HATU and a base such as diisopropylethylamine (DIEA) in a inert polar solvent such as DMF. The carboxylic acids are commercially available or prepared by methods known in the art.

For example, carboxylic acids of formula (IIa) in which R¹ and R² is

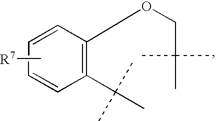

may be prepared by the route illustrated in Reaction Scheme 2.

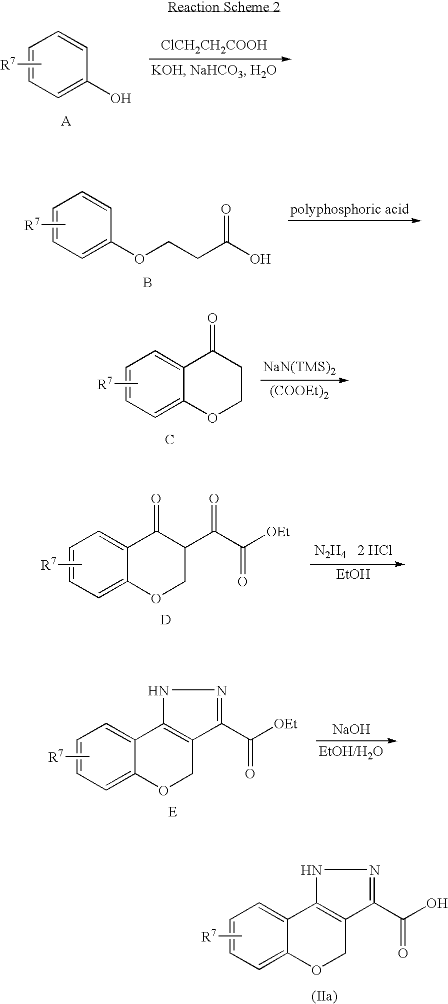

In this scheme, an optionally substituted phenol of formula A is O-alkylated to give the phenoxyalkanoic acid of formula B, which in turn, is cyclized to the chromanone of formula C using an acid catalyst, such as polyphosphoric acid. The intermediate of formula C is then acylated with ethyl oxalate and strong base to give the ketoester of formula D, which upon treatment with hydrazine gives the fused pyrazole ester of formula E. This ester is hydrolyzed to the desired carboxylic acid under basic conditions to provide the compound of formula (IIa).

The salts, e.g., hydrochlorides, of compounds of Formula (I) may be prepared by addition of a non-aqueous. e.g., methanolic, solution of an organic or inorganic acid (e.g., HCl, CF₃CO₂H, etc.) to a solution of the Formula (I) compound and separation of the crystalline salt that is formed. Addition of a non-solvent, such as ether, facilitates the isolate of the salt.

By using these above described methods, the compounds Formula (I) of the invention may be prepared as shown in Table 1 below:

TABLE 1

| Ex. | | |
|---|---|---|
| No. | Structure | NAME |
| 1 | 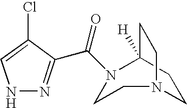 | 4-[(4-chloro-1H-pyrazol-3- yl)carbonyl]-1,4- diazabicyclo[3.2.2]nonane |
| 2 | 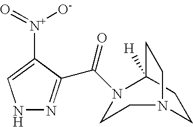 | 4-[(4-nitro-1H-pyrazol-3- yl)carbonyl]-1,4- diazabicyclo[3.2.2]nonane |
| 3 | 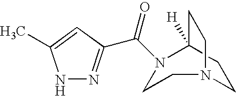 | 4[(5-methyl-1H-pyrazol-3- yl)carbonyl]-1,4- diazabicyclo[3.2.2]nonane |
| 4 | 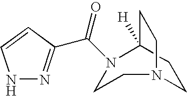 | 4-(1H-pyrazol-3-ylcarbonyl)- 1,4-diazabicyclo[3.2.2]nonane |
| 5 | 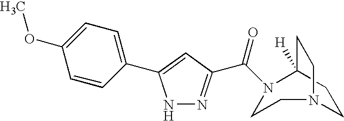 | 4-{[5-(4-methoxyphenyl)-1H- pyrazol-3-yl]carbonyl}-1,4- diazabicyclo[3.2.2]nonane |
| 6 | 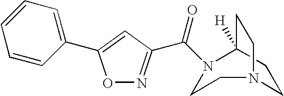 | 4-[(5-phenylisoxazol-3- yl)carbonyl]-1 ,4- diazabicyclo[3.2.2]nonane |
| 7 | 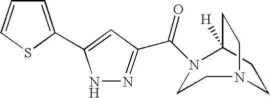 | 4-{[5-(2-thienyl)-1H-pyrazol-3- yl]carbonyl}-1 ,4- diazabicyclo[3.2.2]nonane |
| 8 | 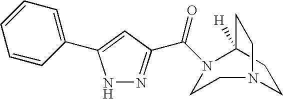 | 4-[(5-phenyl-1H-pyrazol-3- yl)carbonyl]-1,4- diazabicyclo[3.2.2]nonane |
| 9 | 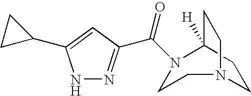 | 4-[(5-cyclopropyl-1H-pyrazol-3- yl)carbonyl]-1,4- diazabicyclo[3.2.2]nonane |
| 10 | 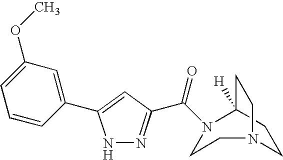 | 4-{[5-(3-methoxyphenyl)-1H- pyrazol-3-yl]carbonyl}-1,4- diazabicyclo[3.2.2]nonane |
| 11 | 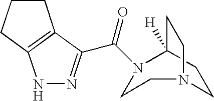 | 4-(1,4,5,6- tetrahydrocyclopenta[c]pyrazol- 3-ylcarbonyl)-1,4- diazabicyclo[3.2.2]nonane |
| 12 | 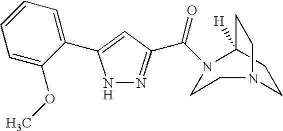 | 4-{[5-(2-methoxyphenyl)-1H- pyrazol-3-yl]carbonyl}-1,4- diazabicyclo[3.2.2]nonane |
| 13 | 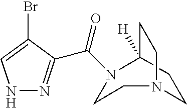 | 4-[(4-bromo-1H-pyrazol-3- yl)carbonyl]-1,4- diazabicyclo[3.2.2]nonane |
| 14 | 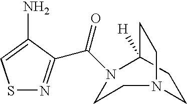 | 3-(1,4-diazabicyclo[3.2.2]non-4- ylcarbonyl)isothiazol-4-amine |
| 15 | 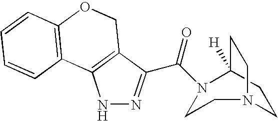 | 3-(1,4-diazabicyclo[3.2.2]non-4- ylcarbonyl)-1,4- dihydrochromeno[4,3-c] pyrazoles |
| 16 | 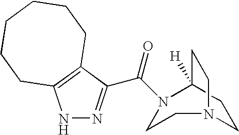 | 3-(1,4-diazabicyclo[3.2.2]non-4- ylcarbonyl)-4,5,6,7,8,9- hexahydro-1H- cycloocta[c]pyrazoles |
| 17 | 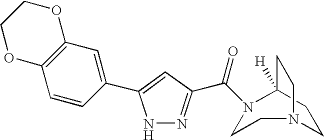 | 4-{[5-(2,3-dihydro-1,4- benzodioxin-6-yl)-1H-pyrazol-3- yl]carbonyl}-1,4- diazabicyclo[3.2.2]nonane |
| 18 | 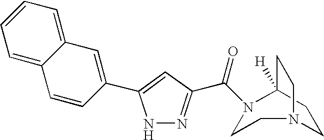 | 4-{[5-(2-naphthyl)-1H-pyrazol-3- yl]carbonyl}-1,4- diazabicyclo[3.2.2]nonane |
| 19 | 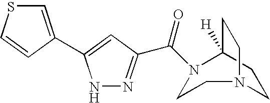 | 4-{[5-(3-thienyl)-1H-pyrazol-3- yl]carbonyl}-1,4- diazabicyclo[3.2.2]nonane |
| 20 | 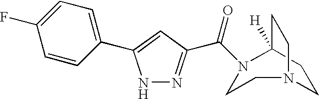 | 4-{[5-(4-fluorophenyl)-1H- pyrazol-3-yl]carbonyl}-1,4- diazabicyclo[3.2.2]nonane |
| 21 | 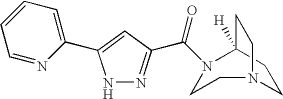 | 4-[(5-pyridin-2-yl-1H-pyrazol-3- yl)carbonyl]-1,4- diazabicyclo[3.2.2]nonane |
| 22 | 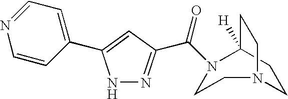 | 4-[(5-pyridin-4-yl-1H-pyrazol-3- yl)carbonyl]-1,4- diazabicyclo[3.2.2]nonane |
| 23 | 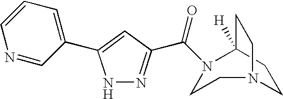 | 4-[(5-pyridin-3-yl-1H-pyrazol-3- yl)carbonyl]-1,4- diazabicyclo[3.2.2]nonane |
| 24 | 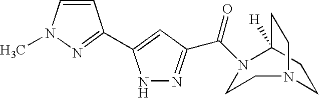 | 5′-(1,4-diazabicyclo[3.2.2]non- 4-ylcarbonyl)-1-methyl-1 H,2′H- 3,3′-bipyrazole |
| 25 | 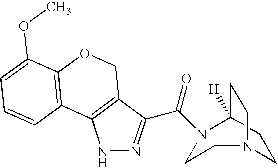 | 3-(1,4-diazabicyclo[3.2.2]non-4- ylcarbonyl)-6-methoxy-1,4- dihydrochromeno[4,3- c]pyrazoles |
| 26 | 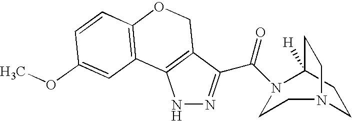 | 3-(1,4-diazabicyclo[3.2.2]non-4- ylcarbonyl)-8-methoxy-1,4- dihydrochromeno[4,3- c]pyrazoles |
| 27 | 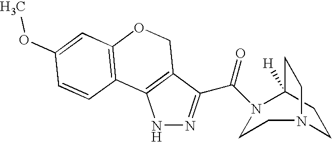 | 3-(1,4-diazabicyclo[3.2.2]non-4- ylcarbonyl)-7-methoxy-1,4- dihydrochromeno[4,3- c]pyrazoles |
| 28 | 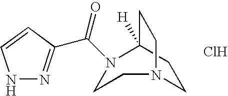 | (1H-pyrazol-3-ylcarbonyl)-1,4- diazabicyclo[3.2.2]nonane hydrochloride |
| 29 | 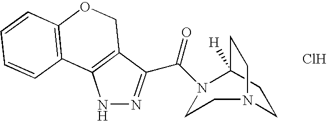 | 3-(1,4-diazabicyclo[3.2.2]non-4- ylcarbonyl)-1,4- dihydrochromeno[4,3- c]pyrazole hydrochloride |
| 30 | 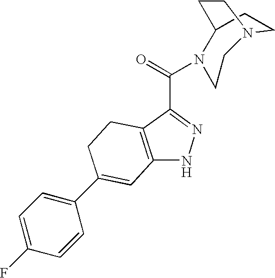 | 3-(1,4-diazabicyclo[3.2.2]non-4- ylcarbonyl)-6-(4-fluorophenyl)- 4,5-dihydro-1H-indazole |
| 31 | 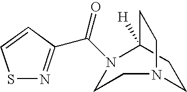 | 4-(isothiazol-3-ylcarbonyl)-1,4- diazabicyclo[3.2.2]nonane |
| 32 | 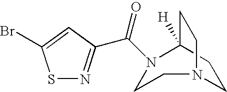 | 4-[(5-bromoisothiazol-3- yl)carbonyl]-1,4- diazabicyclo[3.2.2]nonane |
| 33 | 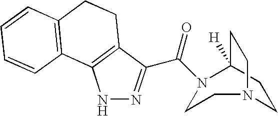 | 3-(1,4-diazabicyclo[3.2.2]non-4- ylcarbonyl)-4,5-dihydro-1H- benzo[g]indazole |
| 34 | 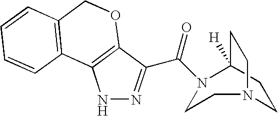 | 3-(1,4-diazabicyclo[3.2.2]non-4- ylcarbonyl)-1,5- dihydroisochromeno[4,3- c]pyrazoles |
| 35 | 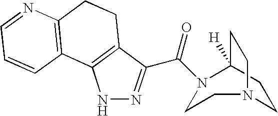 | 3-(1,4-diazabicyclo[3.2.2]non-4- ylcarbonyl)-4,5-dihydro-1H- pyrazolo[3,4-f]quinoline |
| 36 | 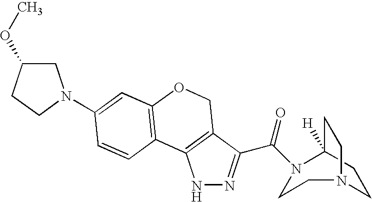 | 3-(1,4-diazabicyclo[3.2.2]non-4- ylcarbonyl)-7-[(3S)-3- methoxypyrrolidin-1-yl]-1,4- dihydrochromeno[4,3- c]pyrazoles |
| 37 | 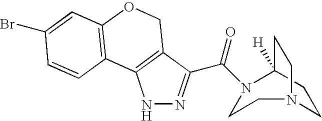 | 7-bromo-3-(1,4- diazabicyclo[3.2.2]non-4- ylcarbonyl)-1,4- dihydrochromeno[4,3- c]pyrazoles |
| 38 | 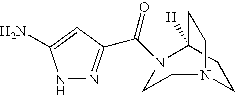 | 3-(1,4-diazabicyclo[3.2.2]non-4- ylcarbonyl)-1H-pyrazol-5-amine |

### Methods of Treatment

Agents that bind to nicotinic acetylcholine receptors have been indicated as useful in the treatment and/or prophylaxis of various diseases and conditions, particularly psychotic diseases, neurodegenerative diseases involving a dysfunction of the cholinergic system, and conditions of memory and/or cognition impairment, including, for example, schizophrenia, anxiety, mania, depression, manic depression [examples of psychotic disorders], Tourette's syndrome, Parkinson's disease, Huntington's disease [examples of neurodegenerative diseases], cognitive disorders (such as Alzheimer's disease, Lewy Body Dementia, Amyotrophic Lateral Sclerosis, memory impairment, memory loss, cognition deficit, attention deficit, Attention Deficit Hyperactivity Disorder), and other uses such as treatment of nicotine addiction, inducing smoking cessation, treating pain (i.e., analgesic use), providing neuroprotection, and treating jetlag. See, e.g., WO 97/30998; WO 99/03850; WO 00142044; WO 01/36417; Holladay et al., J. Med. Chem., 40:26, 4169-94 (1997);

Schmitt et al., Annual Reports Med. Chem., Chapter 5, 41-51 (2000); Stevens et al., Psychopharmatology, (1998) 136: 320-27; and Shytle et al., Molecular Psychiatry, (2002), 7, pp. 525-535.

Thus, in accordance with the invention, there is provided a method of treating a patient, especially a human, suffering from psychotic diseases, neurodegenerative diseases involving a dysfunction of the cholinergic system, and conditions of memory and/or cognition impairment, including, for example, schizophrenia, anxiety, mania, depression, manic depression [examples of psychotic disorders], Tourette's syndrome, Parkinson's disease, Huntington's disease [examples of neurodegenerative diseases], and/or cognitive disorders (such as Alzheimer's disease, Lewy Body Dementia, Amyotrophic Lateral Sclerosis, memory impairment, memory loss, cognition deficit, attention deficit, Attention Deficit Hyperactivity Disorder), comprising administering to the patient an effective amount of a compound according to Formula I.

Neurodegenerative disorders included within the methods of the present invention include, but are not limited to, treatment and/or prophylaxis of Alzheimer's diseases, Pick's disease, diffuse Lewy Body disease, progressive supranuclear palsy

(Steel-Richardson syndrome), multisystem degeneration (Shy-Drager syndrome), motor neuron diseases including amyotrophic lateral sclerosis (ALS), degenerative ataxias, cortical basal degeneration, ALS-Parkinson's-Dementia complex of Guam, subacute sclerosing panencephalitis, Huntington's disease, Parkinson's disease, synucleinopathies, primary progressive aphasia, striatonigral degeneration, Machado-Joseph disease/spinocerebellar ataxia type 3, olivopontocerebellar degenerations, Gilles De La Tourette's disease, bulbar palsy, pseudobulbar palsy, spinal muscular atrophy, spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, prion diseases (such as Creutzfeldt-Jakob, Gerstmann-Straussler-Scheinker disease, Kuru and fatal familial insomnia), and neurodegenerative disorders resulting from cerebral ischemia or infarction including embolic occlusion and thrombotic occlusion as well as intracranial hemorrhage of any type (including, but not limited to, epidural, subdural, subarachnoid and intracerebral), and intracranial and intravertebral lesions (including, but not limited to, contusion, penetration, shear, compression and laceration)

In addition, α7nACh receptor agonists, such as the compounds of the present invention can be used to treat age-related dementia and other dementias and conditions with memory loss including age-related memory loss, senility, vascular dementia, diffuse white matter disease (Binswanger's disease), dementia of endocrine or metabolic origin, dementia of head trauma and diffuse brain damage, dementia pugilistica and frontal lobe dementia. See, e.g., WO 99162505. Thus, in accordance with the invention, there is provided a method of treating a patient, especially a human, suffering from age-related dementia and other dementias and conditions with memory loss comprising administering to the patient an effective amount of a compound according to Formula I.

Thus, in accordance with a further embodiment, the present invention includes methods of treating patients suffering from memory impairment due to, for example, Alzheimer's disease, mild cognitive impairment due to aging, schizophrenia, Parkinson's disease, Huntington's disease, Pick's disease, Creutzfeldt-Jakob disease, depression, aging, head trauma, stroke, CNS hypoxia, cerebral senility, multiinfarct dementia and other neurological conditions, as well as HIV and cardiovascular diseases, comprising administering an effective amount of a compound according to Formula I.

Additionally, the present invention includes methods of treating patients suffering from memory impairment as a result of, for example, chemotherapy, kidney dialysis, post-operative surgery, as well as memory impairment associated with bipolar disorders, comprising administering an effective amount of a compound according to Formula I.

Amyloid precursor protein (APP) and Aβ peptides derived therefrom, e.g., Aβ1-40, Aβ1-42, and other fragments, are known to be involved in the pathology of Alzheimer's disease. The A61-42 peptides are not only implicated in neurotoxicity but also are known to inhibit cholinergic transmitter function. Further, it has been determined that Aβ peptides bind to α7nACh receptors. Thus, agents which block the binding of the Aβ peptides to α-7 nAChRs are useful for treating neurodegenerative diseases. See, e.g., WO 99/62505. In addition, stimulation α7nACh receptors can protect neurons against cytotoxicity associated with Aβ peptides. See, e.g., Kihara, T. et al., Ann. Neural., 1997, 42, 159.

Thus, in accordance with an embodiment of the invention there is provided a method of treating and/or preventing dementia in an Alzheimer's patient which comprises administering to the subject a therapeutically effective amount of a compound according to Formula Ito inhibit the binding of an amyloid beta peptide (preferably, Aβ1-42) with nACh receptors, preferable α7nACh receptors, most preferably, human α7nACh receptors (as well as a method for treating and/or preventing other clinical manifestations of Alzheimer's disease that include, but are not limited to, cognitive and language deficits, apraxias, depression, delusions and other neuropsychiatric symptoms and signs, and movement and gait abnormalities).

The present invention also provides methods for treating other amyloidosis diseases, for example, hereditary cerebral angiopathy, nonneuropathic hereditary amyloid, Down's syndrome, macroglobulinemia, secondary familial Mediterranean fever, Muckle-Wells syndrome, multiple myeloma, pancreatic- and cardiac-related amyloidosis, chronic hemodialysis anthropathy, and Finnish and Iowa amyloidosis.

In addition, nicotinic receptors have been implicated as playing a role in the body's response to alcohol ingestion. Thus, agonists for α7nACh receptors can be used in the treatment of alcohol withdrawal and in anti-intoxication therapy. Thus, in accordance with an embodiment of the invention there is provided a method of treating a patient for alcohol withdrawal or treating a patient with anti-intoxication therapy comprising administering to the patient an effective amount of a compound according to Formula I.

Agonists for the α7nACh receptor subtypes can also be used for neuroprotection against damage associated with strokes and ischemia and glutamate-induced excitotoxicity. Thus, in accordance with an embodiment of the invention there is provided a method of treating a patient to provide for neuroprotection against damage associated with strokes and ischemia and glutamate-induced excitotoxicity comprising administering to the patient an effective amount of a compound according to Formula I.

As noted above, agonists for the α7nACh receptor subtypes can also be used in the treatment of nicotine addiction, inducing smoking cessation, treating pain, and treating jetlag, obesity, diabetes, and inflammation. Thus, in accordance with an embodiment of the invention there is provided a method of treating a patient suffering from nicotine addiction, pain, jetlag, obesity and/or diabetes, or a method of inducing smoking cessation in a patient comprising administering to the patient an effective amount of a compound according to Formula I.

The inflammatory reflex is an autonomic nervous system response to an inflammatory signal. Upon sensing an inflammatory stimulus, the autonomic nervous system responds through the vagus nerve by releasing acetylcholine and activating nicotinic α7 receptors on macrophages. These macrophages in turn release cytokines. Dysfunctions in this pathway have been linked to human inflammatory diseases including rheumatoid arthritis, diabetes and sepsis. Macrophages express the nicotinic α7 receptor and it is likely this receptor that mediates the cholinergic anti-inflammatory response. Therefore, compounds with affinity for the α7nACh receptor on macrophages may be useful for human inflammatory diseases including rheumatoid arthritis, diabetes and sepsis. See, e.g., Czura, C J et al., J. Intern. Med., 2005, 257(2), 156-66.

Thus, in accordance with an embodiment of the invention there is provided a method of treating a patient (e.g., a mammal, such as a human) suffering from inflammation comprising administering to the patient an effective amount of a compound according to Formula I. Similarly, in accordance with another embodiment of the invention there is provided a method of treating a patient (e.g., a mammal, such as a human) suffering from an inflammatory disease, such as, but not limited to, rheumatoid arthritis, diabetes or sepsis, comprising administering to the patient an effective amount of a compound according to Formula I. In accordance with a further embodiment of the invention there is provided a method of treating a patient (e.g., a mammal, such as a human) suffering from inflammatory due to, for example, but not limited to, an autoimmune disease, fibromyalgia, or ulcerative colitis, comprising administering to the patient an effective amount of a compound according to Formula I.

In addition, due to their affinity to α7nACh receptors, labeled derivatives of the compounds of Formula I (e.g., C11 or F18 labeled derivatives), can be used in neuroimaging of the receptors within, e.g., the brain. Thus, using such labeled agents in vivo imaging of the receptors can be performed using, e.g., PET imaging. The condition of memory impairment is manifested by impairment of the ability to learn new information and/or the inability to recall previously learned information. Memory impairment is a primary symptom of dementia and can also be a symptom associated with such diseases as Alzheimer's disease, schizophrenia, Parkinson's disease, Huntington's disease, Pick's disease, Creutzfeldt-Jakob disease, HIV, cardiovascular disease, and head trauma as well as age-related cognitive decline.

Thus, in accordance with an embodiment of the invention there is provided a method of treating a patient suffering from, for example, mild cognitive impairment (MCI), vascular dementia (VaD), age-associated cognitive decline (AACD), amnesia associated w/open-heart-surgery, cardiac arrest, and/or general anesthesia, memory deficits from early exposure of anesthetic agents, sleep deprivation induced cognitive impairment, chronic fatigue syndrome, narcolepsy, AIDS-related dementia, epilepsy-related cognitive impairment, Down's syndrome, Alcoholism related dementia, drug/substance induced memory impairments, Dementia Puglistica (Boxer Syndrome), and animal dementia (e.g., dogs, cats, horses, etc.) comprising administering to the patient an effective amount of a compound according to Formula I.

The dosages of the compounds of the present invention depend upon a variety of factors including the particular syndrome to be treated, the severity of the symptoms, the route of administration, the frequency of the dosage interval, the particular compound utilized, the efficacy, toxicology profile, pharmacokinetic profile of the compound, and the presence of any deleterious side-effects, among other considerations.

The compounds of the invention can be administered to patients, e.g., mammals, particularly humans, at typical dosage levels customary for α-7 nicotinic receptor agonists such as the known α-7 nicotinic receptor agonist compounds mentioned above. For example, the compounds can be administered, in single or multiple doses, by oral administration at a dosage level of, for example, 0.0001-10 mg/kg/day, e.g., 0.01-10 mg/kg/day. Unit dosage forms can contain, for example, 1-200 mg of active compound. For intravenous administration, the compounds can be administered in single or multiple dosages.

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The compounds of the invention also are useful as intermediates for making other compounds of the inventive genus. Thus, for example, compounds exhibiting relatively low activity are also useful for preparing other compounds within the inventive genus.

A compound of Formula (I) can be administered as the sole active agent or in combination with other pharmaceutical agents such as other agents used in the treatment of cognitive impairment and/or memory loss, e.g., other α-7 agonists, PDE4 inhibitors, calcium channel blockers, muscarinic m1 and m2 modulators, adenosine receptor modulators, ampakines, NMDA-R modulators, mGluR modulators, dopamine modulators, serotonin modulators, cannabinoid modulators, cholinesterase inhibitors (e.g., donepezil, rivastigimine, and galanthamine), agents for the treatment of ADHD, anti-depressants, anti-inflammatory agents, anti-psychotic agents (e.g., PDE10 inhibitors), beta secretase modulators, bipolar disorder agents, GABA-nergic drugs, gamma secretase modulators, histamine H3, kinase inhibitors, MAO-B inhibitors, mood stabilizers, 5HT4 modulating agents, 5HT6 antagonists, and α4β2 modulating agents. In such combinations, each active ingredient can be administered either in accordance with their usual dosage range or a dose below their usual dosage range.

The compounds of the invention can be used in conjunction with “positive modulators” which enhance the efficacy of nicotinic receptor agonists. See, e.g., the positive modulators disclosed in WO 99/56745, WO 01/32619, and WO 01/32622. Such combinational therapy can be used in treating conditions/diseases associated with reduced nicotinic transmission.

Further the compounds may be used in conjunction with compounds that bind to Aβ peptides and thereby inhibit the binding of the peptides to α7nAChr subtypes. See, e.g., WO 99/62505.

The present invention further includes methods of treatment that involve activation of α-7 nicotinic receptors. Thus, the present invention includes methods of selectively activating/stimulating α-7 nicotinic receptors in a patient (e.g., a mammal such as a human) wherein such activation/stimulation has a therapeutic effect, such as where such activation may relieve conditions involving neurological syndromes, such as the loss of memory, especially long-term memory. Such methods comprise administering to a patient (e.g., a mammal such as a human), an effective amount of a compound of Formula I alone or as part of a formulation, as disclosed herein. Such co-therapies may be administered in any combination of two or more drugs Such co-therapies may be administered in the form of pharmaceutical compositions, as described below.

### Pharmaceutical Compositions

As used herein, various terms are defined below. When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The term “subject” as used herein includes mammals (e.g., humans and animals).

The term “treatment” includes any process, action, application, therapy, or the like, wherein a subject, including a human being, is provided medical aid with the object of improving the subject's condition, directly or indirectly, or slowing the progression of a condition or disorder in the subject.

The term “combination therapy” or “co-therapy” means the administration of two or more therapeutic agents to treat a condition and/or disorder. Such administration encompasses co-administration of two or more therapeutic agents in a substantially simultaneous manner, such as in a single capsule having a fixed ratio of active ingredients or in multiple, separate capsules for each inhibitor agent. In addition, such administration encompasses use of each type of therapeutic agent in a sequential manner. Additionally, each therapeutic agent may be administered using the same or different modes of administration.

The phrase “therapeutically effective” means the amount of each agent administered that will achieve the goal of improvement in conditions or severity of disorders associated with defective or malfunctioning nicotinic acetylcholine receptors, while avoiding or minimizing adverse side effects associated with the given therapeutic treatment.

The term “pharmaceutically acceptable” means that the subject item is appropriate for use in a pharmaceutical product.

Based on well known assays used to determine the efficacy for treatment of conditions identified above in mammals, and by comparison of these results with the results of known medicaments that are used to treat these conditions, the effective dosage of Formula (I) compounds(s) can readily be determined for treatment of each desired indication. The amount of the active ingredient (e.g., a compound of Formula (I)) to be administered in the treatment of one of these conditions can vary widely according to such considerations as the particular compound and dosage unit employed, the mode of administration, the period of treatment, the age and sex of the patient treated, and the nature and extent of the condition treated.

Formula (I) compounds for use in methods of the invention may be administered as compound per se. Alternatively, a compound of Formula (I) may be administered with an acceptable carrier in the form of a pharmaceutical composition. The pharmaceutically acceptable carrier must be compatible with the other ingredients of the composition and must not be intolerably deleterious to the recipient. The carrier can be a solid or a liquid, or both, and preferably is formulated with the compound as a unit-dose composition, for example, a tablet, which can contain from about 0.05% to about 95% by weight of the active compound(s) based on a total weight of the dosage form. Other pharmacologically active substances can also be present, including other compounds useful in the treatment of a condition associated with defective or malfunctioning nicotinic acetylcholine receptors.

A compound of Formula (I) for use in methods of the present invention may be administered by any suitable route, preferably in the form of a pharmaceutical composition adapted to such a route, and in a therapeutically effective dose for the treatment intended. A compound of Formula (I) may, for example, be administered orally, sublingually, nasally, pulmonary, mucosally, parenterally, intravascularly, intraperitoneally, subcutaneously, intramuscularly or topically. Unit dose formulations, particularly orally administrable unit dose formulations such as tablets or capsules, generally contain, for example, from about 0.001 to about 500 mg, preferably from about 0.005 mg to about 100 mg, and more preferably from about 0.01 to about 50 mg, of the active ingredient. In the case of pharmaceutically acceptable salts, the weights indicated above for the active ingredient refer to the weight of the pharmaceutically active ion derived from the salt.

Of course, the specific initial and continuing dosage regimen to prevent, treat, give relief from, or ameliorate a condition or disorder associated with defective or malfunctioning nicotinic acetylcholine receptors, or to otherwise protect against or treat these conditions for each patient will vary according to the nature and severity of the condition as determined by the attending diagnostician, the activity of the specific Formula (I) compound employed, the age of the patient, the diet of the patient, time of administration, route of administration, rate of excretion of the drug, drug combinations, pharmacological considerations such as the activity, efficacy, pharmacokinetics and toxicology profiles of the particular Formula (I) inhibitor employed, whether a drug delivery system is utilized, and whether the Formula (I) compound is administered with other active ingredients, and the like. The desired mode of treatment and number of doses of an α7nAChR inhibitor may be ascertained by those skilled in the art using conventional treatment tests.

The Formula (I) compound may be utilized to achieve the desired pharmacological effect by administration to a patient in need thereof in an appropriately formulated pharmaceutical composition. A patient, for the purpose of this invention, is a mammal, including a human, in need of treatment for a particular condition or disease. Therefore, the present invention includes pharmaceutical compositions which are comprised of a pharmaceutically acceptable carrier and a therapeutically effective amount of the Formula (I) compound. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. The Formula (I) compound may be administered with a pharmaceutically acceptable carrier using any effective conventional dosage unit forms, including, for example, immediate and timed release preparations, orally, parenterally, topically, or the like.

For oral administration, the Formula (I) compound may be formulated into solid or liquid preparations such as, for example, capsules, pills, tablets, troches, lozenges, melts, powders, solutions, pastes, syrups, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch. The pharmaceutical composition is preferably made in the form of a dosage unit containing a particular amount of the active ingredient.

The Formula (I) compound may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and/or one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents. Oral delivery of the Formula (I) compound(s) can include formulations well known in the art to provide immediate delivery or prolonged or sustained delivery of a drug to the gastrointestinal tract by any number of mechanisms. Immediate delivery formulations include, but are not limited to, oral solutions, oral suspensions, fast-dissolving tablets or capsules, sublingual tablets, disintegrating tablets and the like. Prolonged or sustained delivery formulations include, but are not limited to, pH sensitive release of the active ingredient from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. The intended effect is to extend the time period over which an active drug molecule is delivered to the site of action by manipulation of the dosage forma Thus, enteric-coated and enteric-coated controlled release formulations may be used in methods of the present invention. Suitable enteric coatings include cellulose acetate phthalate, polyvinylacetate phthalate, hydroxypropylmethyl-cellulose phthalate and anionic polymers of methacrylic acid and methacrylic acid methyl ester.

Pharmaceutical compositions can be prepared by any suitable method of pharmacy, which includes the step of bringing into association, the Formula (I) compound and the carrier (which can constitute one or more accessory ingredients). In general, the compositions are prepared by uniformly and intimately admixing the Formula (I) compound with a liquid or finely divided solid carrier, or both, and then, if necessary, shaping the product. For example, a tablet can be prepared by compressing or molding a powder or granules of the inhibitors, optionally with one or more accessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made, for example, by molding the powdered compound in a suitable machine.

Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Pharmaceutical compositions suitable for buccal (sub-lingual) administration include lozenges comprising a Formula (I) compound in a flavored base, usually sucrose, and acacia or tragacanth, and pastilles comprising the inhibitors in an inert base such as gelatin and glycerin or sucrose and acacia.

The Formula (I) compound(s) may also be administered. parenterally, that is, subcutaneously, intravenously, intramuscularly, or interperitoneally, as injectable dosages of the compound in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the invention may also be administered in the form of suppositories for rectal administration of the drug. These compositions may be prepared by mixing the drug (e.g., the Formula (I) compound) with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

As noted above, a compound of Formula (I) can be administered as the sole active agent or in combination with other pharmaceutical agents. Thus, the pharmaceutical compositions according to the invention may further comprise at least additional active agent, such as other agents used in the treatment of cognitive impairment and/or memory loss, e.g., other α-7 agonists, PDE4 inhibitors, calcium channel blockers, muscarinic m1 and m2 modulators, adenosine receptor modulators, ampakines, NMDA-R modulators, mGluR modulators, dopamine modulators, serotonin modulators, cannabinoid modulators, cholinesterase inhibitors (e.g., donepezil, rivastigimine, and galanthamine), agents for the treatment of ADHD, anti-depressants, anti-inflammatory agents, anti-psychotic agents (e.g., PDE10 inhibitors), beta secretase modulators, bipolar disorder agents, GABA-nergic drugs, gamma secretase modulators, histamine H3, kinase inhibitors, MAO-B inhibitors, mood stabilizers, 5HT4 modulating agents, 5HT6 antagonists, and α4β2 modulating agents. In such combinations, each active ingredient can be administered either in accordance with their usual dosage range or a dose below their usual dosage range.

The pharmaceutical composition according to the invention may further comprise at least one “positive modulator” which enhances the efficacy of nicotinic receptor agonists. See, e.g., the positive modulators disclosed in WO 99156745, WO 01/32619, and WO 01/32622. Such combinational therapy can be used in treating conditions/diseases associated with reduced nicotinic transmission.

The pharmaceutical composition according to the invention may further comprise at least one compound that binds to Aβ peptides and thereby inhibit the binding of the peptides to α7nAChr subtypes. See, e.g., WO 99/62505.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, trolamine.

Other pharmaceutical ingredients include, for example, but are not limited to, adsorbents (e.g., powdered cellulose and activated charcoal); aerosol propellants (e.g., carbon dioxide, CCl₂F₂, F₂ClC—CClF₂ and CClF₃); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange. No. 5, D&C Red No. 8, caramel and ferric oxide red); clarifying agents (e.g., bentonite); emulsifying agents (but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, oxtoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrillin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

The total daily dose of each inhibitor can be administered to the patient in a single dose, or in multiple subdoses. Typically, subdoses can be administered two to six times per day, preferably two to four times per day, and even more preferably two to three times per day. Doses can be in immediate release form or sustained release form sufficiently effective to obtain the desired control over the condition associated with defective or malfunctioning nicotinic acetylcholine receptors.

Formula (I) compound(s) may also be utilized in compositions, in research and diagnostics, or as analytical reference standards, and the like. Therefore, the present invention includes compositions which are comprised of an inert carrier and an effective amount of the Formula (I) compound. An inert carrier is any material which does not interact with a compound to be carried and which lends support, means of conveyance, bulk, traceable material, and the like to the compound to be carried. An effective amount of compound is that amount which produces a result or exerts an influence on the particular procedure being performed.

A Formula (I) compound for use in methods of the invention may also be administered as the pharmaceutically acceptable salt, protected acid, conjugate acid, tautomer, prodrug or stereoisomer of a compound found to have α7nAChR stimulating, activating or inhibiting activity. Tautomers include, for example, hydroxy tautomers. Protected acids include, but are not limited to, protected acids such as esters, hydroxyamino derivatives, amides and sulfonamides. Formation of prodrugs is well known in the art in order to enhance the properties of the parent compound; such properties include solubility, absorption, biostability and release time (see “Pharmaceutical Dosage Form and Drug Delivery Systems” (Sixth Edition), edited by. Ansel et al., publ. by Williams & Wilkins, pgs. 27-29, (1995) which is hereby incorporated by reference). Commonly used prodrugs are designed to take advantage of the major drug biotransformation reactions and are also to be considered within the scope of the invention. Major drug biotransformation reactions include N-dealkylation, O-dealkylation, aliphatic hydroxylation, aromatic hydroxylation, N-oxidation, S-oxidation, deamination, hydrolysis reactions, glucuronidation, sulfation and acetylation (see Goodman and Gilman's The Pharmacological Basis of Therapeutics (Ninth Edition), editor Molinoff et al., publ. by McGraw-Hill, pages 11-13, (1996), which is hereby incorporated by reference).

Besides being useful for human treatment, administration of a Formula (I) compound may also be useful for veterinary treatments of companion animals (e.g., horses, dogs, cats, etc.), exotic animals and farm animals. Even though the invention is described in terms of human biology, it is understood by those of ordinary skill in the art that the present invention is applicable to other mammals as well.

Formulations suitable for subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20^{th} edition, 2000).

The present invention will now be further described by way of the following non-limiting examples. In applying the disclosure of these examples, it should be kept clearly in mind that other and different embodiments of the methods disclosed according to the present invention will no doubt suggest themselves to those of skill in the relevant art.

In the foregoing and in the following examples, all temperatures are set forth uncorrected in degrees Celsius; and, unless otherwise indicated, all parts and percentages are by weight.

The entire disclosures of all applications, patents and publications, cited above and below, are hereby incorporated by reference.

### Abbreviations and Acronyms

A comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference. For purposes of this invention, the chemical elements are identified in accordance with the Periodic Table of the Elements, CAS version, Handbook of Chemistry and Physics, 67th Ed., 1986-87.

More specifically, when the following abbreviations are used throughout this disclosure, they have the following meaning:
Ac acetyl
AcOH acetic acid

AIBN azobisisobutyronitrile
amu atomic mass unit
aq aqueous
Bu butyl
CDI carbonyl diimidazole
Celite® brand of diatomaceous earth filtering agent, registered trademark of Celite Corporation
conc concentrated
d doublet
doublet of doublet
ddd doublet of doublet of doublet
DIBAL diisobutylaluminum hydride
DIPA diisopropylamine
DME dimethyoxyethane
N,N-dimethyl formamide
DMSO dimethylsulfoxide
DMSO-d₆ dimethylsulfoxide-d₆
dppf 1,1′-bis(diphenylphosphino)ferrocene
EDCI 1-(3-dimethylaminopropyl)-3-ethylcarbodiimide hydrochloride
electron impact ionization
EI-MS electron impact-mass spectrometry
equiv equivalent
ES MS electrospray mass spectrometry
Et ethyl
Et₂O diethyl ether
Et₃N triethylamine
EtOAc ethyl acetate
EtOH ethanol
Ex example
g gram
GC-MS gas chromatography-mass spectrometry
h hour(s)
HATU O-(7-Azabenzotriazol-1-yl)-N,N,N′,N′-tetramethyluronium hexafluorophosphate
HBTU O-(Benzotriazol-1-yl)-N,N,N,N′-tetramethyluronium hexafluorophosphate
Hex hexanes
[3H] MLA tritiated methyllycaconitine citrate
¹H NMR proton nuclear magnetic resonance
HOAT 1-hydroxy-7-aza-benzotriazole
HOBT 1-hydroxybenzotriazole
HPLC high-performance liquid chromatography
HPLC ES-MS high-performance liquid chromatography-electrospray mass spectroscopy
Int intermediate
KOtBu potassium tert-butoxide
L liter
LCMS liquid chromatography/mass spectroscopy
m multiplet
M molar
mL milliliter
m/z mass over charge
Me methyl
MeCN acetonitrile
MeOH methanol
mg milligram
MHz megahertz
min minute(s)
mmol millimole
mol mole
mp melting point
MS mass spectrometry
N normal
NaOAc sodium acetate
NBS N-bromosuccinimide
NMM 4-methylmorpholine
¹H NMR nuclear magnetic resonance
Pd₂(dba)₃ tris(dibenzylideneacetone)dipalladium(0)
Pd(OAc)₂ palladium acetate
Pd(PPh₃)₄ tetrakis(triphenylphosphine)palladium(0)
Pd/C palladium on carbon
Pd(dppf)Cl₂ [1,1′-bis(diphenylphosphino)ferrocene]dichloropalladium(II)
Ph phenyl
ppm parts per million
Pr propyl
psi pounds per square inch
q quartet
qt quintet
R_{f} TLC retention factor
rt room temperature
s singlet
t triplet
t_{R} retention time (HPLC)
TBAF tetrabutylammonium fluoride
TBSCl tert-butyldimethylsilyl chloride
TBS tert-butyldimethylsilyl
TBTU O-(Benzotriazol-1-yl)-N,N,N,N′-tetramethyluronium tetrafluoroborate
TFA trifluoroacetic acid
THF tetrahydrofuran
TLC thin layer chromatography
TMS tetramethylsilane
pTSA 4-methylbenzenesulfonic acid (p-toluenesulfonic acid)
v/v volume per unit volume
vol volume
w/w weight per unit weight

The following specific examples are presented to further illustrate the invention described herein, but they should not be construed as limiting the scope of the invention in any way.

### Experimental Examples

### General

¹HNMR spectra are recorded at 300 MHz on a Bruker Instruments NMR unless otherwise stated. Coupling constants (J) are in Hertz (Hz) and peaks are listed relative to TMS (δ 0.00 ppm).

Sulfonic acid ion exchange resins (SCX) are purchased from Varian Technologies.

Analytical HPLC//MS is performed on Waters Micromass using 4.6 mm×100 mm Xterra RP₁₈ 3.5μ columns using (i) a gradient of 20/80 to 80/20 acetonitrile (0.1% formic acid)/water (0.1% formic acid) over 8 min (Method A), or (ii) a gradient of 5/95 to 80/20 acetonitrile (0.1% formic acid)/water (0.1% formic acid) over 8 min (Method B). Preparative HPLC is performed on 20 mm×250 mm 5μ SHISEIDO, CAPCELL PAK C18 column using a gradient of 90/10 to 0/100 water/acetonitrile over 25 minutes while monitoring λ254 nm in the UV spectrum.

Many of the carboxylic acids used in the experimental are commercially available. The procedure to prepare the carboxylic acids that are not commercially available is provided below. 1,4-Diazabicyclo[3.2.2]nonane dihydrochloride was purchased from Olainfarm. Hydrochloride salts of the bicycle amides are prepared by adding an ethereal solution of hydrochloric acid to a methanolic solution of the bicyclic amide, followed by isolation of the resulting precipitate.

Other carboxylic acids used in the experimental are prepared by straightforward synthetic methods known to those skilled in the art, for example, using the method illustrated in Procedures 1-5 described below:

### Procedure 1.

The following procedure describes the preparation of substituted and modified dihydrochromen-4-ones from phenols:

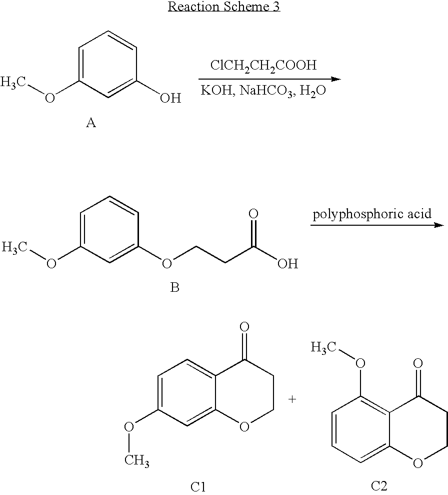

### Dihydrochromen-4-one Synthesis (Ketone Synthesis 1)

A solution of 2-methoxyphenol (40.3 mmol) in potassium hydroxide (15 mL) is added to a solution of 3-chloropropanoic acid (40.7 mmol) and sodium bicarbonate (40.5 mmol) in water (15 mL). The reaction mixture is heated at 110° C. for 16 h and is allowed to cool to rt. The pH of the reaction mixture is adjusted.to 5 with 10% aqueous hydrochloric acid and is extracted with ether (3×100 mL). The combined organic layers are extracted with sodium bicarbonate (3×80 mL) and the pH of the combined aqueous layers is adjusted to 5 with 10% aqueous hydrochloric acid. The aqueous layer is extracted with dichloromethane (150 mL), dried (sodium sulfate), and concentrated to provide 3-(2-methoxyphenoxy)propanoic acid (Target B) in 14% yield as a yellow solid.

3-(2-Methoxyphenoxy)propanoic acid (5.61 mmol) and polyphosphoric (25 mL) are combined and heated at 67° C. for 1 h. The reaction mixture is diluted with ice water (150 mL) and the precipitated solids are collected by filtration, washed with water, and dried to provide 8-methoxy-2,3-dihydrochromen-4-one (Target C2) in 67% yield as a brown solid.

The following dihydrochromen-4-ones are prepared using this procedure, starting from the appropriately substituted phenols:
8-Methoxy-2,3-dihydrochromen-4-one.
7-Methoxy-2,3-dihydrochromen-4-one.
6-Methoxy-2,3-dihydrochromen-4-one.
7-Bromo-2,3-dihydrochromen-4-one.
3,4-Dihydronaphthalen-1(2H)-one is commercially available.

### Procedure 2.

The following procedure describes the preparation of tetrahydroquinolin-5-one from cyclohexane-1,3-dione:

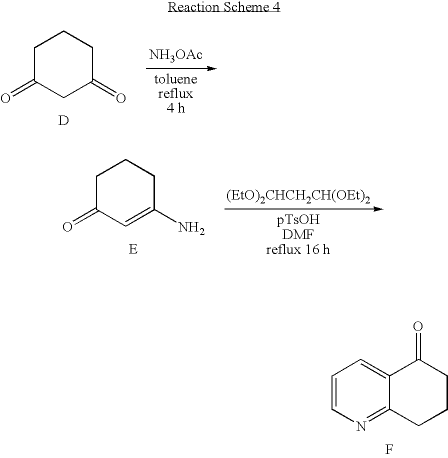

### Tetrahydroquinolin-5-one Synthesis (Ketone Synthesis 2)

Ammonium acetate (151 mmol) and cyclohexane-1,3-dione (150.00 rnmol) are combined and diluted with toluene (300 mL) in a round bottom flask with a Dean-Stark condenser. The reaction mixture is heated at reflux for 4 h and is concentrated. The solid residue is recrystallized (ethyl acetate) to provide 3-amino-2-cyclohexen-1-one as a yellow solid. The material is of sufficient purity to use in the subsequent transformation.

A mixture of 3-aminocyclohex-2-enone (100 mmol), 1,1,3,3-tetraethoxypropane (110 mmol), and 4-methylbenzenesulfonic acid (2.91 mmol) is diluted with N,N-dimethylformamide (40 mL) and the reaction mixture is heated at reflux for 16 h. The reaction mixture is allowed to cool to rt, neutralized with sodium bicarbonate, diluted with water (400 mL), and is extracted with ethyl acetate (3×100 mL). The combined organic layers are dried (sodium sulfate) and concentrated. The residue is purified by chromatography (10/1 petroleum ether/ethyl acetate) to provide 5,6,7,8-tetrahydroquinolin-5-one in 7% yield as a colorless oil.

The following tetrahydroquinolin-5-one is prepared using this method: 5,6,7,8-Tetrahydroquinolin-5-one.

### Procedure 3.

The following procedure describes the synthesis of isochromen-4(3H)-ones from 2-methylbenzonitriles:

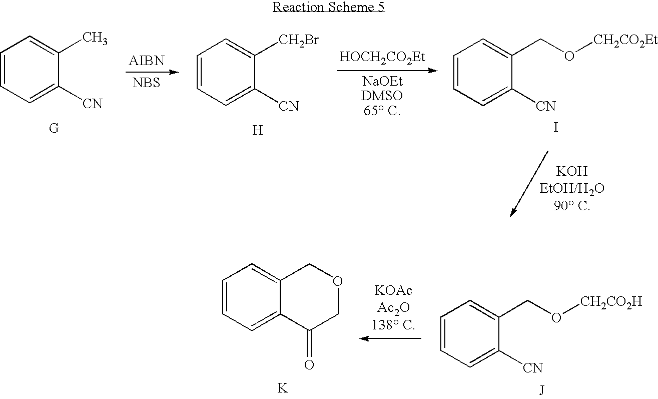

### 1H-Isochromen-4(3H)-one Synthesis (Ketone Synthesis 3)

Azobisisobutyronitrile (AIBN, 29.25 mmol) is added to a solution of 2-methylbenzonitrile (325 mmol) and N-bromosuccinimide (NBS, 346 mmol) in carbontetrachloride (300 mL). The reaction mixture is heated at 90° C. for 2 h and is allowed to cool to rt. The precipitated solids are removed by filtration and the filtrate is washed with saturated aqueous sodium bicarbonate (4×120 mL), dried (sodium sulfate), and concentrated. The resulting solid is washed with hexane (4×500 mL) and dried to provide 2-(bromomethyl)benzonitrile in 57% yield as a yellow solid. A solution of 2-(bromomethyl)benzonitrile (214 mmol) in dimethylsulfoxide (40 mL) is added over 90 min to a solution of ethyl 2-hydroxyacetate (431 mmol) and sodium ethoxide (215 mmol) in dimethylsulfoxide (14.5 mL). The reaction mixture is maintained at it for 1 h, is heated at 65° C. for 5 h, and is allowed to cool to rt. The reaction mixture is diluted with ice water (50 g) and extracted with ether (3×500 mL) and the combined organic layers are dried (sodium sulfate), and concentrated. The residue is purified by chromatography (100/1 to 50/1 petroleum ether/ethyl acetate) to provide ethyl [(2-cyanobenzyl)oxy]acetate as a yellow oil.

Potassium hydroxide (309 mmol) is added to a solution of ethyl [(2-cyanobenzyl)oxy]acetate (62.5 mmol) in ethanol (60 mL) and water (60 mL). The reaction mixture is heated at 90° C. for 16 h, allowed to cool to rt, and the pH adjusted to ˜1 with concentrated hydrochloric acid. The resulting solution is extracted with dichloromethane (3×100 mL) and the combined organic layers are dried (sodium sulfate), and concentrated to provide crude 2-[(carboxymethoxy)methyl]benzoic acid as a brown solid. The material is of sufficient purity to use in the subsequent step. 2-[(Carboxymethoxy)methyl]benzoic acid (37.6 mmol) and potassium acetate (169.2 mmol) are combined and diluted with acetic anhydride (112 mL). The reaction mixture is heated at 138° C. for 2 h and is concentrated. The residue is diluted with ice water (25 g), extracted with ether (3×900 mL) and the combined organic layers are dried (magnesium sulfate), and concentrated. The residue is diluted with ethanol (150 mL) and 4 N sodium hydroxide (20 mL) and the reaction mixture is maintained for 2 h at rt. The reaction mixture is extracted with ether (3×900 mL) and the combined organic layers are dried (magnesium sulfate), and concentrated. The residue is purified by chromatography (100/1 to 30/1 hexane/ethyl acetate) to provide 1H-isochromen-4(3H)-one in 47% yield as a yellow oil.

The following isochromenone is prepared using this procedure:

1H-Isochromen-4(3H)-one

### Procedure 4.

The following procedure describes the preparation of pyrazole carboxylic acids, starting from ketones, either commercially available or prepared as described above by Procedures 1-3:

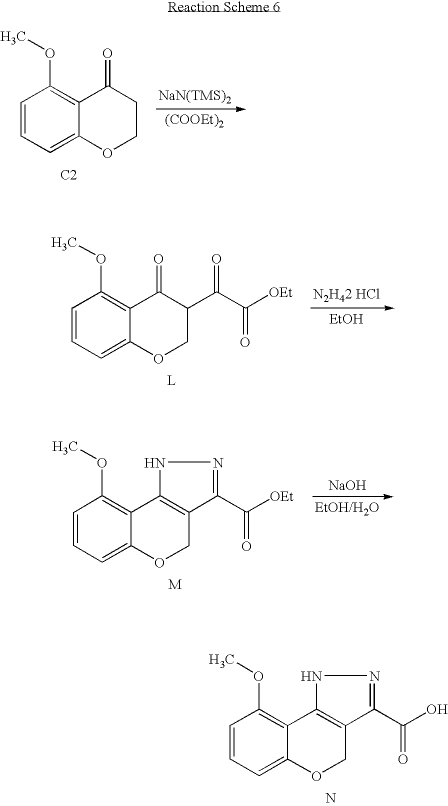

Dihydrochromeno[4,3-c]pyrazole acid Synthesis

Sodium hexamethyldisilazide (1 M in tetrahydrofuran, 4.02 mmol) is added dropwise to a solution of 8-methoxy-2,3-dihydrochromen-4-one (C2, 3.93 mmol) in tetrahydrofuran (20 mL) at −78° C. and the reaction mixture is maintained for 30 min Diethyl oxalate (4.02 mmol) is added dropwise and the reaction mixture is allowed to warm to it and maintained for 1 h. The reaction mixture is diluted with 10% aqueous hydrochloric acid (20 mL) and is extracted with ethyl acetate (150 mL). The organic layer is dried (magnesium sulfate) and concentrated to provide ethyl 2-(8-methoxy-4-oxo-3,4-dihydro-2H-chromen-3-yl)-2-oxoacetate (Target L) in 96% yield as yellow oil.

Hydrazine hydrate (4.00 mmol) is added to a solution of ethyl 2-(8-methoxy-4-oxo-3,4-dihydro-2H-chromen-3-yl)-2-oxoacetate (L, 3.96 mmol) in ethanol (40 mL) and the reaction mixture is heated at reflux for 1 h. The reaction mixture is diluted with ice water (140 mL), extracted with ethyl acetate (200 mL), dried (sodium sulfate), and concentrated to provide ethyl 6-methoxy-1,4-dihydrochromeno[4,3-c]pyrazole-3-carboxylate (Target M) in 65% yield as a yellow solid.

A solution of sodium hydroxide (12.8 mmol) in water (10 mL) is added to a solution of ethyl 6-methoxy-1,4-dihydrochromeno[4,3-c]pyrazole-3-carboxylate (M, 2.43 mmol) in ethanol (100 mL). The reaction mixture is heated at reflux for 3 h and is concentrated. The residue is diluted with water (100 mL) and is extracted with ethyl acetate (200 mL). The pH of the aqueous layer is adjusted to 2-3 with 10% aqueous hydrochloric acid and the precipitated solids are collected by filtration, washed with water and hexane, and dried to provide 6-methoxy-1,4-dihydrochromeno[4,3-c]pyrazole-3-carboxylic acid (Target N) in 64% yield as a yellow solid. ¹H-NMR (DMSO-d₆): δ 7.2 (1H, d), 6.9 (2H, m), 5.4 (2H, s), 3.7 (3H, s); LC/MS (ES, m/z) [M+1]+247.

The following substituted dihydrochromeno[4,3-c]pyrazole acids are prepared using this method, starting from the appropriate ketone:
6-Methoxy-1,4-dihydrochromeno[4,3-c]pyrazole-3-carboxylic acid.
7-Methoxy-1,4-dihydrochromeno[4,3-c]pyrazole-3-carboxylic acid
7-Bromo-1,4-dihydrochromeno[4,3-c]pyrazole-3-carboxylic acid.
8-Methoxy-1,4-dihydrochromeno[4,3-c]pyrazole-3-carboxylic acid.
4,5-Dihydro-1H-benzo[g]indazole-3-carboxylic acid.
4,5-Dihydro-1H-pyrazolo[3,4-f]quinoline-3-carboxylic acid.
1,5-Dihydroisochromeno[4,3-c]pyrazole-3-carboxylic acid.

### Procedure 5.

The following procedure describes a method to prepare 6-(4-fluorophenyl)-4,5-dihydro-1H-indazole-3-carboxylic acid

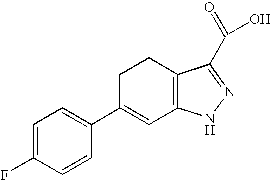

A solution of 3-ethoxy-2-cyclohexen-1-one (15.0 mmol) in tetrahydrofuran (7.5 mL) is added over 10 min to a solution of 4-fluorophenylmagnesium bromide (1.0 M in tetrahydrofuran, 15.0 mL) in tetrahydrofuran (7.5 mL) at −5° C. The reaction mixture is maintained for 30 min, is allowed to warm to rt and is maintained for an additional 2 h. The solution is poured into 1 N aqueous hydrochloric acid and is maintained for 1 h. The reaction mixture is diluted with water (50 mL) and extracted with ethyl acetate (50 mL). The organic layer is washed with brine (25 mL), dried (magnesium sulfate), and concentrated. The residue is purified by chromatography (90/10 to 65/35 hexanes/ethyl acetate) to provide 3-(4-fluorophenyl)cyclohex-2-en-1-one in 49% yield.

A solution of 3-(4-fluorophenyl)cyclohex-2-en-1-one (7.40 mmol) in ether (10.0 mL) is added to a solution of lithium hexamethyldisilazide (1.00 M in tetrahydrofuran, 8.0 mL) in ether (10.0 mL) at −78° C. After 15 min, a solution of ethanedioic acid, dimethyl ester (11.0 mmol) in ether (10.0 mL) is added and the reaction mixture is allowed to warm to it and is maintained for 16 h. The reaction mixture is partitioned between water (50 mL) and ethyl acetate (50 mL) and is neutralized with 1N hydrochloric acid. The layers are separated and the organic layer is washed with brine (25 mL), dried (magnesium sulfate), and concentrated to provide a yellow solid. The solid is recrystallized (ethanol) to provide methyl [4-(4-fluorophenyl)-2-oxocyclohex-3-en-1-yl](oxo)acetate in73% yield as a light yellow solid.

Hydrazine (3.80 mmol) is added to a solution of methyl [4-(4-fluorophenyl)-2-oxocyclohex-3-en-1-yl](oxo)acetate (2.70 mmol) in ethanol (10.0 mL) and t reaction mixture is heated at reflux for 30 min. The mixture is allowed to cool to it and the precipitated solids are collected by filtration to give methyl 6-(4-fluorophenyl)-4,5-dihydro-1H-indazole-3-carboxylate in 80% yield as an off-white solid. The acid is prepared by standard saponification conditions (sodium hydroxide in ethanol/water) and used without further purification.

The following acid is prepared using this method:
6-(4-Fluorophenyl)-4,5-dihydro-1H-indazole-3-carboxylic acid.

### Procedure 6.

The following procedure describes the synthesis of isothiazole-3-carboxylic acid from 5-amino-3-methylisothiazole hydrochloride.

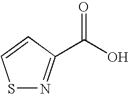

Sodium nitrite (36.4 mmol) is added in several batches to a solution of 5-amino-3-methylisothiazole hydrochloride (33.1 mmol) in sulfuric acid (25 mL) at 0° C. A solution of copper(II) oxide (1.67 mmol) in 30% phosphoric acid (70 mL) is added dropwise to the solution and the reaction mixture is maintained at 0° C. for 1 h. The reaction mixture is warmed to 40° C. and is maintained for an additional 1 h. The pH of the reaction mixture is adjusted to ˜9 with 25% sodium hydroxide and is extracted with ether (3×100 mL). The combined organic layers are dried (sodium sulfate) and concentrated to provide 3-methylisothiazole in 12% yield as yellow oil.

Chromium(IV) oxide (9.00 mmol) is added in several batches to a solution of 3-methylisothiazole (3.03 mmol) in fuming sulfuric acid (10 mL) at 0° C. The reaction mixture is allowed to warm to rt and is maintained for 16 h. The reaction mixture is diluted with ice water (100 mL), extracted with ether (6×200 mL) and the combined organic layers are dried (sodium sulfate) and concentrated. The residue is purified by preparative HPLC to provide isothiazole-3-carboxylic acid in 13% yield as a white solid. ¹H-NMR (400MHz, DMSO-d₆) δ 3.43 (s, 1H), 9.17 (d, 1H), 7.80 (d, 1H), 3.43 (s, 1H); LC/MS (ES, m/z) [M+1]⁺ 128.

The following acid is prepared using this method:
Isothiazole-3-carboxylic acid.

### Procedure 7.

The following procedure describes the preparation of 5-bromoisothiazole-3-carboxylic acid from 5-amino-3-methylisothiazole hydrochloride.

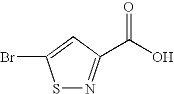

A solution of sodium nitrite (66.7 mmol) in water (6 mL) is added dropwise to a solution of 3-methylisothiazol-5-amine hydrochloride (66.2 mmol) in phosphoric acid (25 mL) and nitric acid (13 mL) at 0° C. and the reaction mixture is maintained for 30 min. A solution of copper(I) bromide (66.2 mmol) in concentrated hydrobromic acid (50 mL) is added dropwise and the reaction mixture is maintained at 0° C. for 60 min when the pH of the solution is adjusted to ˜4 with 2 N sodium hydroxide (100 mL). The resulting solution is extracted with ether (3×200 mL) and the combined organic layers are dried (sodium sulfate) and concentrated. The solid residue is recrystallized from petroleum ether/ethyl acetate to provide 5-bromo-3-methylisothiazole in 13% yield as a yellow oil.

Chromium(IV) oxide (21.9 mmol) is added in several batches to a solution of 5-bromo-3-methylisothiazole (7.30 mmol) in fuming sulfuric acid (30 mL) at 0° C. The reaction mixture is allowed to warm to it and is maintained for 16 h. The reaction mixture is diluted with ice water (100 mL), extracted with ether (3×200 mL) and the combined organic layers are dried (sodium sulfate) and concentrated. The residue is purified by preparative HPLC to provide 5-bromoisothiazole-3-carboxylic acid in 13% yield as a white solid. ¹H-NMR (DMSO-d₆): δ 13.8 (broad s, 1H), 7.9 (s, 1H); LC/MS (ES, m/z) [M]⁺ 208

The following acid is prepared using this procedure:
5-Bromoisothiazole-3-carboxylic acid.

Hydrochloride salts of the bicycle amides are prepared by adding an ethereal solution of hydrochloric acid to a methanolic solution of the bicyclic amide, followed by isolation of the resulting precipitate.

### Procedure 8.

The following procedure describes the synthesis of 7-[(3S)-3-methoxypyrrolidin-1-yl]-1,4-dihydrochromeno[4,3-c]pyrazole-3-carboxylic acid hydrochloride from ethyl 7-bromo-1,4-dihydrochromeno[4,3-c]pyrazole-3-carboxylate.

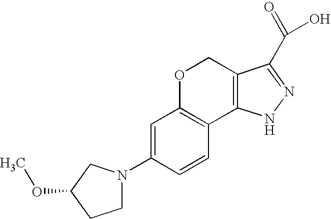

3,4-Dihydro-2H-pyran (11.9 mmol) and 4-methylbenzenesulfonic acid (0.58 mmol) are added to a solution of ethyl 7-bromo-1,4-dihydrochromeno[4,3-c]pyrazole-3-carboxylate (9.29 mmol) in dichloromethane (100 mL) and tetrahydrofuran (20 mL) and the reaction mixture is maintained at it for 16 h. The reaction mixture is washed with water (3×100 mL), dried (sodium sulfate), and concentrated to provide ethyl 7-bromo-1-(tetrahydro-2H-pyran-2-yl)-1,4-dihydrochromeno[4,3-c]pyrazole-3-carboxylate in 43% yield as a brown solid.

A mixture of ethyl 7-bromo-1-(tetrahydro-2H-pyran-2-yl)-1,4-dihydrochromeno[4,3-c]pyrazole-3-carboxylate (3.98 mmol), (S)-3-methoxypyrrolidine (8.02 mmol), BINAP (0.48 mmol), cesium carbonate (10.1 mmol),and palladium(II) acetate (0.40 mmol) under an atmosphere of nitrogen is diluted with toluene (100 mL). The reaction mixture is heated at 110° C. for 16 h and is concentrated. The residue is diluted with water (50 mL) and is extracted with ethyl acetate (3×300 mL) and the combined organic layers are dried (sodium sulfate) and concentrated. The residue is purified by chromatography (20/1 to 10/1 petroleum ether/ethyl acetate) to provide ethyl 7-[(3S)-3-methoxypyrrolidin-1-yl]-1-(tetrahydro-2H-pyran-2-yl)-1,4-dihydrochromeno[4,3-c]pyrazole-3-carboxylate in 59% yield as a white solid.

A solution of sodium hydroxide (10.0 mmol) in water (5 mL) is added to a solution of ethyl 7-[(3S)-3-methoxypyrrolidin-1-yl]-1-(tetrahydro-2H-pyran-2-yl)-1,4-dihydrochromeno[4,3-c]pyrazole-3-carboxylate (1.87 mmol) in ethanol (35 mL) and the reaction m mixture is heated at 90° C. for 1 h. The reaction mixture is concentrated, diluted with water (35 mL), and extracted with ethyl acetate (3×300 mL). The pH of the combined aqueous layers is adjusted to ˜3 with 10% aqueous hydrochloric acid and is concentrated to provide 7-[(3S)-3-methoxypyrrolidin-1-yl]-1-(tetrahydro-2H-pyran-2-yl)-1,4-dihydrochromeno[4,3-c]pyrazole-3-carboxylic acid in 45% yield as a white solid. Gaseous hydrochloric acid is bubbled through a solution of 7-[(3S)-3-methoxypyrrolidin-1-yl]-1-(tetrahydro-2H-pyran-2-yl)-1,4-dihydrochromeno[4,3-c]pyrazole-3-carboxylic acid (0.85 mmol) in 1,4-dioxane (60 mL) for 30 min. The reaction mixture is maintained for 2 h at rt and is concentrated. The crude product is purified by preparative HPLC to provide 7-[(3S)-3-methoxypyrrolidin-1-yl]-1,4-dihydrochromeno[4,3-c]pyrazole-3-carboxylic acid hydrochloride in 19% yield as a yellow solid.

The following acid is prepared using this method:
7-[(3S)-3-methoxypyrrolidin-1-yl]-1,4-dihydrochromeno[4,3-c]pyrazole-3-carboxylic acid hydrochloride.

### Representative Preparative Examples of the Invention Compounds

### Example 1

### Preparation of 4-[(4-Chloro-1H-pyrazol-3-yl)carbonyl]-1,4-diazabicyclo[3.2.2]nonane

### (Representative Procedure A)

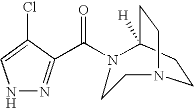

The following provides a general method for the coupling of bicyclobases and carboxylic acids to form carboxamide derivatives.

A solid mixture of 4-chloro-1H-pyrazole-3-carboxylic acid (1.47 mmol), N,N,N′,N′-tetramethyl-O-(7-azabenzotriazol-1-yl)uronium hexafluorophosphate (HATU) (1.06 mmol), and 1,4-diazabicyclo[3.2.2]nonane dihydrochloride (0.979 mmol) is diluted with N,N-dimethylformamide (6.0 mL) and N,N-diisopropylethylamine (5.7 mmol) and the reaction mixture is maintained for 16 h at rt. The reaction mixture is transferred to a SCX column (10 g) and flushed with 5 volumes of methanol. The partially purified product is then eluted using 2.0 M ammonia in methanol and concentrated. The residue is purified by gradient preparative chromatography, starting from 100/0 to 50/50 ratio mixture of solvent A/solvent B, where A=ethyl acetate and B is (50/50/2) ethyl acetate/methanol/dimethylethylamine, to provide the product in 64% yield as an off-white solid.

¹H NMR (CD₃OD) δ [3/1 rotamer mixture] 7.80 (s, 1H), 4.71 (m, 0.75H), 4.10 (m, 0.25H), 3.98 (m, 0.5H), 3.68 (m, 1.5H), 3.09-2.98 (m, 6H), 2.17-1.84 (four m, 4H). LC/MS (EI) t_{R} 1.45 min (Method A), m/z 255.1.min.

Using Procedure A described above for Example 1, the following carboxamide derivative compounds of Examples 2-38 can be similarly prepared and are characterized below:

### Example 2

### 4-[(4-Nitro-1H-pyrazol-3-yl)carbonyl]-1,4-diazabicyclo[3.2.2]nonane

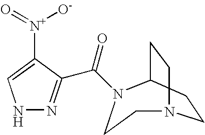

The compound is prepared as described for Example 1 starting from 1,4-diazabicyclo[3.2.2]nonane dihydrochloride and 4-nitro-1H-pyrazole-3-carboxylic acid: LC/MS (EI) t_{R} 3.90 min (Method B), m/z 266.1.

### Example 3

### 4-[(5-Methyl-1H-pyrazol-3-yl)carbonyl]-1,4-diazabicyclo[3.2.2]nonane

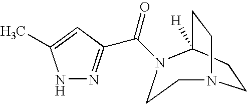

The compound is prepared as described for Example 1 starting from 1,4-diazabicyclo[3.2.2]nonane dihydrochloride and 5-methyl-1H-pyrazole-3-carboxylic acid: LC/MS (EI) t_{R} 1.42 min (Method A), m/z 235.1.

### Example 4

### 4-(1H-Pyrazol-3-ylcarbonyl)-1,4-diazabicyclo[3.2.2]nonane

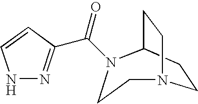

The compound is prepared as described for Example 1 starting from 1,4-diazabicyclo[3.2.2]nonane dihydrochloride and 1H-pyrazole-3-carboxylic acid: LC/MS (EI) t_{R} 4.40 min (Method B), m/z 221.1.

### Example 5

### 4-([5-(4-Methoxyphenyl)-1H-pyrazol-3-yl]carbonyl}-1,4-diazabicyclo[3.2.2]nonane

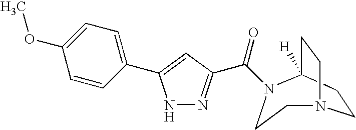

The compound is prepared as described for Example 1 starting from 1,4-diazabicyclo[3.2.2]nonane dihydrochloride and 5-(4-methoxyphenyl)-1H-pyrazole-3-carboxylic acid: LC/MS (EI) t_{R} 2.58 min (Method A), m/z 327.1.

### Example 6

### 4-[(5-Phenylisoxazol-3-yl)carbonyl]-1,4-diazabicyclo[3.2.2]nonane

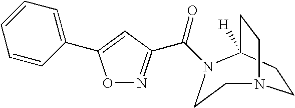

The compound is prepared as described for Example 1 starting from 1,4-diazabicyclo[3.2.2]nonane dihydrochloride and 5-phenylisoxazole-3-carboxylic acid: LC/MS (EI) t_{R} 3.53 min (Method A), m/z 298.1.

### Example 7

### 4-{[5-(2-Thienyl)-1H-pyrazol-3-yl]carbonyl}-1,4-diazabicyclo[3.2.2]nonane

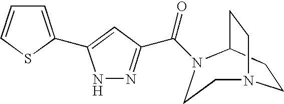

The compound is prepared as described for Example 1 starting from 1,4-diazabicyclo[3.2.2]nonane dihydrochloride and 5-(2-thienyl)-1H-pyrazole-3-carboxylic acid: LC/MS (EI) t_{R} 2.54 min (Method A), m/z 303.

### Example 8

### 4-[(5-phenyl-1H-pyrazol-3-yl)carbonyl]-1,4-diazabicyclo[3.2.2]nonane

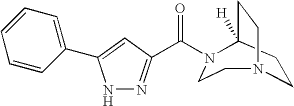

The compound is prepared as described for Example 1 starting from 1,4-diazabicyclo[3.2.2]nonane dihydrochloride and 5-phenyl-1H-pyrazole-3-carboxylic acid: LC/MS (EI) t_{R} 2.58 min (Method A), m/z 297.1.

### Example 9

### 4-[(5-Cyclopropyl-1H-pyrazol-3-yl)carbonyl]-1,4-diazabicyclo[3.2.2]nonane

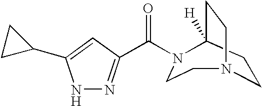

The compound is prepared as described for Example 1 starting from 1,4-diazabicyclo[3.2.2]nonane dihydrochloride and 5-cyclopropyl-1H-pyrazole-3-carboxylic acid: LC/MS (EI) t_{R} 1.55 min (Method A), m/z 261.2.

### Example 10

### 4-{[5-(3-Methoxyphenyl)-1H-pyrazol-3-yl]carbonyl}-1,4-diazabicyclo[3.2.2]nonane

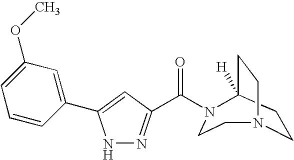

The compound is prepared as described for Example 1 starting from 1,4-diazabicyclo[3.2.2]nonane dihydrochloride and 5-(3-methoxyphenyl)-1H-pyrazole-3-carboxylic acid: LC/MS (EI) t_{R} 2.64 min (Method A), m/z 327.2.

### Example 11

### 4-(1,4,5,6-Tetrahydrocyclopenta[c]pyrazol-3-ylcarbonyl)-1,4-diazabicyclo[3.2.2]nonane

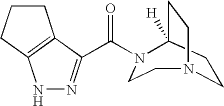

The compound is prepared as described for Example 1 starting from 1,4-diazabicyclo[3.2.2]nonane dihydrochloride and 1,4,5,6-tetrahydrocyclopentapyrazole-3-carboxylic acid: LC/MS (EI) t_{R} 1.52 min (Method A), m/z 261.2.

### Example 12

### 4-{[5-(2-Methoxyphenyl)-1H-pyrazol-3-yl]carbonyl}-1,4-diazabicyclo[3.2.2]nonane

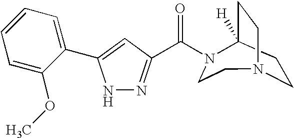

The compound is prepared as described for Example 1 starting from 1,4-diazabicyclo[3.2.2]nonane dihydrochloride and 5-(2-methoxyphenyl)-1H-pyrazole-3-carboxylic acid:

LC/MS (EI) t_{R} 2.63 min (Method A), m/z 327.2.

### Example 13

### 4-[(4-Bromo-1H-pyrazol-3-yl)carbonyl]-1,4-diazabicyclo[3.2.2]nonane

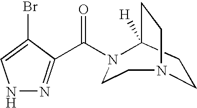

The compound is prepared as described for Example 1 starting from 1,4-diazabicyclo[3.2.2]nonane dihydrochloride and 4-bromo-1H-pyrazole-3-carboxylic acid: LC/MS (EI) t_{R} 1.47 min (Method A), m/z 299/301.

### Example 14

### 3-(1,4-Diazabicyclo[3.2.2]non-4-ylcarbonyl)isothiazol-4-amine

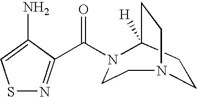

The compound is prepared as described for Example 1 starting from 1,4-diazabicyclo[3.2.2]nonane dihydrochloride and 4-aminoisothiazole-3-carboxylic acid: LC/MS (EI) t_{R} 1.41 min (Method A), m/z 253.1.

### Example 15

### 3-(1,4-Diazabicyclo[3.2.2]non-4-ylcarbonyl)-1,4-dihydrochromeno[4,3-c]pyrazole

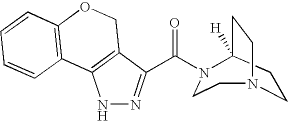

The compound is prepared as described for Example 1 starting from 1,4-diazabicyclo[3.2.2]nonane dihydrochloride and 1,4-dihydrochromeno[4,3-c]pyrazole-3-carboxylic acid: LC/MS (EI) t_{R} 2.77 min (Method A), m/z 325.1.

### Example 16

### 3-(1,4-Diazabicyclo[3.2.2]non-4-ylcarbonyl)-4,5,6,7,8,9-hexahydro-1H-cycloocta[c]pyrazole

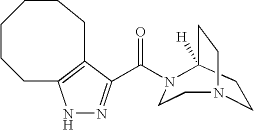

The compound is prepared as described for Example 1 starting from 1,4-diazabicyclo[3.2.2]nonane dihydrochloride and 4,5,6,7,8,9-hexahydro-1H-cyclooctapyrazole-3-carboxylic acid: LC/MS (EI) t_{R} 2.54 min (Method A), m/z 303.1.

### Example 17

### 4-{[5-(2,3-Dihydro-1,4-benzodioxin-6-yl)-1H-pyrazol-3-yl]carbonyl}-1,4-diazabicyclo[3.2.2]nonane

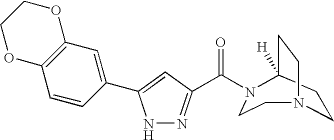

The compound is prepared as described for Example 1 starting from 1,4-diazabicyclo[3.2.2]nonane dihydrochloride and 5-(2,3-dihydrobenzo[1,4]dioxin-6-yl)-1H-pyrazole-3-carboxylic acid: LC/MS (EI) t_{R} 2.54 min (Method A), m/z 355.1

### Example 18

### 4-{[5-(2-Naphthyl)-1H-pyrazol-3-yl]carbonyl}-1,4-diazabicyclo[3.2.2]nonane

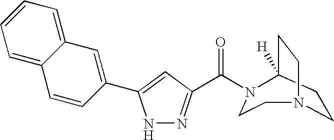

The compound is prepared as described for Example 1 starting from 1,4-diazabicyclo[3.2.2]nonane dihydrochloride and 5-naphthalen-2-yl-1H-pyrazole-3-carboxylic acid: LC/MS (EI) t_{R} 3.79 min (Method A), m/z 347.2.

### Example 19

### 4-{[5-(3-Thienyl)-1H-pyrazol-3-yl]carbonyl}-1,4-diazabicyclo[3.2.2]nonane

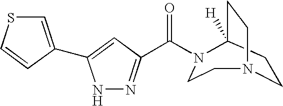

The compound is prepared as described for Example 1 starting from 1,4-diazabicyclo[3.2.2]nonane dihydrochloride and 5-thiophen-3-yl-1H-pyrazole-3-carboxylic acid: LC/MS (EI) t_{R} 2.54 min (Method A), m/z 303.1.

### Example 20

### 4-{[5-(4-Fluorophenyl)-1H-pyrazol-3-yl]carbonyl}-1,4-diazabicyclo[3.2.2]nonane

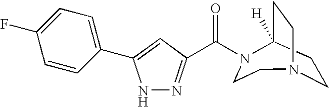

The compound is prepared as described for Example 1 starting from 1,4-diazabicyclo[3.2.2]nonane dihydrochloride and 5-(4-fluorophenyl)-1H-pyrazole-3-carboxylic acid: LC/MS (EI) t_{R} 2.63 min (Method A), m/z 315.1.

### Example 21

### 4-[(5-Pyridin-2-yl-1H-pyrazol-3-yl)carbonyl]-1,4-diazabicyclo[3.2.2]nonane

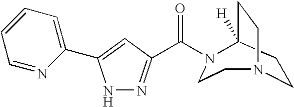

The compound is prepared as described for Example 1 starting from 1,4-diazabicyclo[3.2.2]nonane dihydrochloride and 5-pyridin-2-yl-1H-pyrazole-3-carboxylic acid: LC/MS (EI) t_{R} 1.42 min (Method A), m/z 298.2.

### Example 22

### 4-[(5-Pyridin-4-yl-1H-pyrazol-3-yl)carbonyl]-1,4-diazabicyclo[3.2.2]nonane

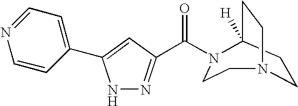

The compound is prepared as described for Example 1 starting from 1,4-diazabicyclo[3.2.2]nonane dihydrochloride and 5-pyridin-4-yl-1H-pyrazole-3-carboxylic acid: LC/MS (EI) t_{R} 1.35 min (Method A), m/z 298.1

### Example 23

### 4-[(5-Pyridin-3-yl-1H-pyrazol-3-yl)carbonyl]-1,4-diazabicyclo[3.2.2]nonane

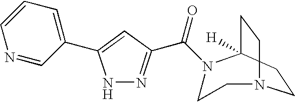

The compound is prepared as described for Example 1 starting from 1,4-diazabicyclo[3.2.2]nonane dihydrochloride and 5-pyridin-3-yl-1H-pyrazole-3-carboxylic acid: LC/MS (EI) t_{R} 1.40 min (Method A), m/z 298.1.

### Example 24

### 5′-(1,4-Diazabicyclo[3.2.2]non-4-ylcarbonyl)-1-methyl-1H,2′-3,3′-bipyrazole

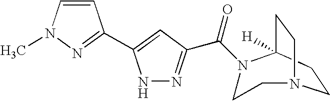

The compound is prepared as described for Example 1 starting from 1,4-diazabicyclo[3.2.2]nonane dihydrochloride and 1′-methyl-2H,1′H-[3,3]bipyrazolyl-5-carboxylic acid: LC/MS (EI) t_{R} 1.45 min (Method A), m/z 301.2.

### Example 25

### 3-(1,4-Diazabicyclo[3.2.2]non-4-ylcarbonyl)-6-methoxy-1,4-dihydrochromeno[4,3-c]pyrazole

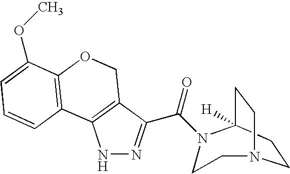

The compound is prepared as described for Example 1 starting from 1,4-diazabicyclo[3.2.2]nonane dihydrochloride and 6-methoxy-1,4-dihydrochromeno[4,3-c]pyrazole-3-carboxylic acid: LC/MS (EI) t_{R} 2.55 min (Method A), m/z 355.2.

### Example 26

### 3-(1,4-Diazabicyclo[3.2.2]non-4-ylcarbonyl)-8-methoxy-1,4-dihydrochromeno[4,3-c]pyrazole

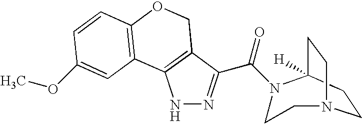

The compound is prepared as described for Example 1 starting from 1,4-diazabicyclo[3.2.2]nonane dihydrochloride and 8-methoxy-1,4-dihydrochromeno[4,3-c]pyrazole-3-carboxylic acid: LC/MS (EI) t_{R} 2.94 min (Method A), m/z 355.2.

### Example 27

### 3-(1,4-Diazabicyclo[3.2.2]non-4-ylcarbonyl)-7-methoxy-1,4-dihydrochromeno[4,3-c]pyrazole

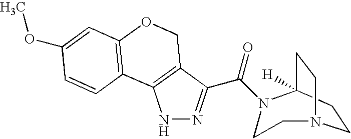

The compound is prepared as described for Example 1 starting from 1,4-diazabicyclo[3.2.2]nonane dihydrochloride and 8-methoxy-1,4-dihydrochromeno[4,3-c]pyrazole-3-carboxylic acid: LC/MS (EI) t_{R} 2.87 min (Method A), m/z 355.2.

### Example 28

### (1H-Pyrazol-3-ylcarbonyl)-1,4-diazabicyclo[3.2.2]nonane Hydrochloride

### (Representative Procedure B)

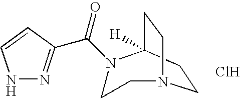

The following provides a general method for the production of salts of the bicyclobase adducts created using Representative Procedure A.

4-(1H-Pyrazol-3-ylcarbonyl)-1,4-diazabicyclo[3.2.2]nonane is prepared from 1H-pyrazole-3-carboxylic acid and 1,4-diazabicyclo[3.2.2]nonane dihydrochloride using Example 1 in 71% yield. The amide (0.645 mmol) is dissolved in methanol (5 mL) and treated with 1 M hydrochloric acid in ether (7 mL). The reaction mixture is maintained for 2 h and is diluted with ether (15 mL) to induce more crystallization. The solids are isolated and recrystallized from methanol/ethyl acetate to provide the salt in 46% yield as a colorless solid.

¹HNMR: CD₃OD δ 7.76 (s, 1H), 6.73 (s, 1H), 5.20 (broad s, 1H), 4.47 (m, 1H), 4.18 (m, 1H), 3.64-3.52 (m, 7H), 2.35 (m, 2H), 2.18 (M, 2H).

Using Procedure B described above for Example 28, the following compound of Examples 29 can be similarly prepared and is characterized below:

### Example 29

### 3-(1,4-Diazabicyclo[3.2.2]non-4-ylcarbonyl)-1,4-dihydrochromeno[4,3-c]pyrazole hydrochloride

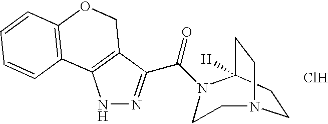

The compound is prepared as described for Example 28 starting from the compound prepared in Example 15: LC/MS (EI) t_{R} 2.82 min (Method A), m/z 325.1.

### Example 30

### 3-(1,4-Diazabicyclo[3.2.2]non-4-ylcarbonyl)-6-(4-fluorophenyl)-4,5-dihydro-1H-indazole

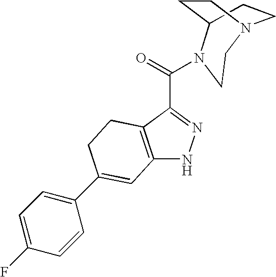

The compound is prepared as described for Example 1 starting from 6-(4-fluorophenyl)-4,5-dihydro-1H-indazole-3-carboxylic acid: LC/MS (EI) t_{R} 3.83 (Method A), m/z 367.2.

### Example 31

### 4-(Isothiazol-3-ylcarbonyl)-1,4-diazabicyclo[3.2.2]nonane

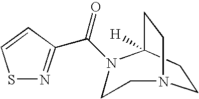

The compound is prepared as described for Example 1 starting from isothiazole-3-carboxylic acid: LC/MS (EI) t_{R} 1.47 min (Method A), m/z 238.1.

### Example 32

### 4-[(5-Bromoisothiazol-3-yl)carbonyl]-1,4-diazabicyclo[3.2.2]nonane

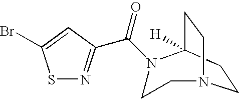

The compound is prepared as described for Example 1 starting from 5-bromoisothiazole-3-carboxylic acid: LC/MS (EI) t_{R} 2.55 (Method A), m/z 316.01318.0.

### Example 33

### 3-(1,4-Diazabicyclo[3.2.2]non-4-ylcarbonyl)-4,5-dihydro-1H-benzo[g]indazole

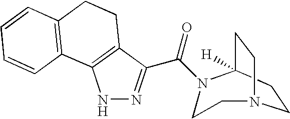

The compound is prepared as described for Example 1 starting from 4,5-dihydro-1H-benzo[g]indazole-3-carboxylic acid: LC/MS (EI) t_{R} 2.53 min (Method A), m/z 323.2.

### Example 34

### 3-(1,4-Diazabicyclo[3.2.2]non-4-ylcarbonyl)-1,5-dihydroisochromeno[4,3-c]pyrazole

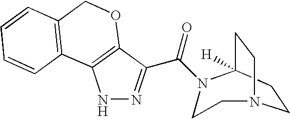

The compound is prepared as described for Example 1 starting from 1,5-dihydroisochromeno[4,3-c]pyrazole-3-carboxylic acid LC/MS (EI) t_{R} 2.42 min (Method A), m/z 325.

### Example 35

### 3-(1,4-Dazabicyclo[3.2.2]non-4-ylcarbonyl)-4,5-dihydro-1H-pyrazolo[3,4-f]quinoline

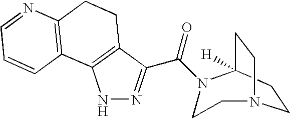

The compound is prepared as described for Example 1 starting from 4,5-dihydro-1H-pyrazolo[3,4-f]quinoline-3-carboxylic acid: LC/MS (EI) t_{R} 1.27 min (Method A), m/z 324.1.

### Example 36

### 3-(1,4-Dazabicyclo[3.2.2]non-4-ylcarbonyl)-74(3S)-3-methoxypyrrolidin-1-yl]-1,4-dihydrochromeno[4,3-c]pyrazole

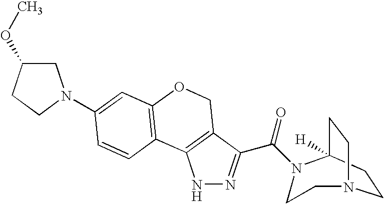

The compound is prepared as described for Example 1 starting from 7-[(3S)-3-methoxypyrrolidin-1-yl]-1,4-dihydrochromeno[4,3-c]pyrazole-3-carboxylic acid: LC/MS (EI) t_{R} 3.38 min (Method A), m/z 424.2.

### Example 37

### 7-Bomo-3-(1,4-diazabicyclo[3.2.2]non-4-ylcarbonyl)-1,4-dihydrochromeno[4,3-c]pyrazole

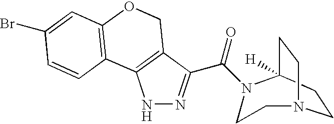

The compound is prepared as described for Example 1 starting from 7-bromo-1,4-dihydrochromeno[4,3-c]pyrazole-3-carboxylic acid LC/MS (EI) t_{R} 3.87 min (Method A), m/z 402.1/405.0.

### Example 38

### 3-(1,4-Dzabicyclo[3.2.2]non-4-ylcarbonyl)-1H-pyrazol-5-amine

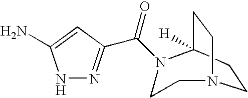

The compound is prepared as described for Example 1 starting from 5-aminopyrazole-3-carboxylic acid LC/MS (EI) t_{R} 1.42 min (Method A), m/z 236.1.

### Example 39

### Evaluation of Compounds with a [3H] MLA Binding Assay

The procedure for [³H]MLA binding assay is the same as described in WO2004/029050 A1, except the receptor resource is humanized monkey α7 receptors (See WO 03/095976).

### Materials:

Humanized monkey α7 receptors

Protease inhibitor cocktail tablet: Roche, CAT No. 1697498

### Membrane Preparation

Rat brains in 20 vol (wlv) of ice-cold 0.32 M sucrose with protease inhibitors (one tablet per 50 ml,) are homogenized with a polytron for 10 sec at setting 11, then centrifuged 10 min at 1000 g, 4° C. The supernatant is centrifuged again for 20 min at 20,000 g, 4° C. The pellets are resuspended in binding buffer (200 mM TRIS-HCl, 20 mM HEPES, pH 7.5, 144 mM NaCl, 1.5 mM KCl, 1 mM MgSO₄, 2 mM CaCl₂, 0.1% (wlv) BSA) and stored membrane prep at −80° C.

For saturation assay, the 200 μl assay mixture in binding buffer contains 200 μg of membrane protein, 0.2 to 44 nM of [3H] MLA. The nonspecific binding is defined using 1 μM MLA. Competition assay is carried out with 2 nM [3H] MLA and a desirable range of compounds. The assay mixture is incubated at 22° C. for 2 hours. Binding affinities for the preferred compounds of the invention are 3 nM to 10 μM.

### Capsule Formulation

A capsule formula is prepared from:

| | |
|---|---|
| Formula (I) compound | 10 mg |
| Starch | 109 mg |
| Magnesium stearate | 1 mg |

The components are blended, passed through an appropriate mesh sieve, and filled into hard gelatin capsules.

### Tablet Formulation

A tablet is prepared from:

| | | |
|---|---|---|
| Formula (I) compound | 25 | mg |
| Cellulose, microcrystaline | 200 | mg |
| Colloidal silicon dioxide | 10 | mg |
| Stearic acid | 5.0 | mg |

The ingredients are mixed and compressed to form tablets. Appropriate aqueous and non-aqueous coatings may be applied to increase palatability, improve elegance and stability or delay absorption.

### Sterile IV Solution

A mg/mL solution of the Formula (I) compound is made using sterile, injectable water, and the pH is adjusted if necessary. The solution is diluted for administration with sterile 5% dextrose and is administered as an IV infusion.

### Intramuscular Suspension

The following intramuscular suspension is prepared:

| | | |
|---|---|---|
| Formula (I) compound | 50 | μg/mL |
| Sodium carboxymethylcellulose | 5 | mg/mL |
| TWEEN 80 | 4 | mg/mL |
| Sodium chloride | 9 | mg/mL |
| Benzyl alcohol | 9 | mg/mL |

The suspension is administered intramuscularly.

### Hard Shell Capsules

A large number of unit capsules are prepared by filling standard two-piece hard galantine capsules each with powdered active ingredient, 150 mg of lactose, 50 mg of cellulose, and 6 mg of magnesium stearate.

### Soft Gelatin Capsules

A mixture of active ingredient in a digestible oil such as soybean oil, cottonseed oil, or olive oil is prepared and injected by means of a positive displacement pump into molten gelatin to form soft gelatin capsules containing the active ingredient. The capsules are washed and dried. The active ingredient can be dissolved in a mixture of polyethylene glycol, glycerin and sorbitol to prepare a water miscible medicine mix.

### Immediate Release Tablets/Capsules

These are solid oral dosage forms made by conventional and novel processes.

These units are taken orally without water for immediate dissolution and delivery of the medication. The active ingredient is mixed in a liquid containing ingredient such as sugar, gelatin, pectin, and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

The preceding examples can be repeated with similar success by substituting the generically or specifically described reactants and/or operating conditions of this invention for those used in the preceding examples.

While the invention has been illustrated with respect to the production of particular compounds, it is readily apparent to those of ordinary skill in the art that variations and modifications of the invention can be made without departing from the spirit or scope of the invention.

All publications and patents mentioned in the above specification are incorporated herein by reference.

Various modifications and variations of the described methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of diabetes or related fields are intended to be within the scope of the following claims. Those skilled in the art will recognize, or be able to ascertain using no more than routine experimentation, many equivalents to the specific embodiments of the invention described herein. Such equivalents are intended to be encompassed by the following claims.

We claim:

1. A compound according to of Formula (I): 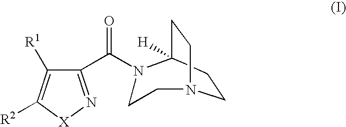
wherein
X is NH, N(CH₃), S or O;
R¹ and R² are each, independently, hydrogen, C₁-C₆-alkyl which is unsubstituted or substituted one or more times by R¹⁰, C₂-C₆-alkenyl which is unsubstituted or substituted one or more times by R¹⁰, C₂-C₆-alkynyl which is unsubstituted or substituted one or more times by R¹⁰, C₃-C₆-cycloalkyl which is unsubstituted or substituted one or more times by R¹¹, C₃-C₈-cycloalkenyl which is unsubstituted or substituted one or more times by R¹¹, halo, OR³, SR³, NR³R⁴, aryl which is unsubstituted or substituted one or more times by R¹², heterocyclyl which is unsubstituted or substituted one or more times by R¹², S(O)ₚR¹³, S(O)ₚNR³R⁴, —C(O)R³, —C(O)OR³, —C(O)NR³R⁴, NO₂, or CN, or
R¹ and R² taken together are 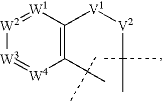
—(CH₂)₂CR⁹═CR⁹—, or —(CH₂)ₘ;
R³ and R⁴ are each, independently, hydrogen, C₁-C₆-alkyl which is unsubstituted or substituted one or more times by R¹⁰, C₃-C₆-alkenyl which is unsubstituted or substituted one or more times by R¹⁰, C₃-C₆-alkynyl which is unsubstituted or substituted one or more times by R¹⁰, C₃-C₆-cycloalkyl which is unsubstituted or substituted one or more times by R¹¹, C₃-C₈-cycloalkenyl which is unsubstituted or substituted one or more times by R¹¹, aryl which is unsubstituted or substituted one or more times by R¹², heterocyclyl which is unsubstituted or substituted one or more times by R¹², —C(O)R⁵, —C(O)OR⁵, or —C(O)NR⁵R⁶;
W¹, W², W³ and W⁴ are each, independently, CR⁷ or N, wherein no more than one of W¹, W², W³ and W⁴ is N;
V¹ and V² are each, independently, O, CR⁸R⁸, S, NH, or NR³, provided that when one of V¹ or V² represent O, S, NH, or NR³, the other is CR⁸R⁸;
m is 3, 4, 5, or 6;
n is 0, 1 or 2;
p is 1 or 2;
R⁵ and R⁶ are each, independently, hydrogen, C₁-C₆-alkyl, C₃-C₆-alkenyl, C₃-C₆-alkynyl, C₃-C₆-cycloalkyl, C₃-C₈-cycloalkenyl, aryl, or heterocyclyl;
R⁷ is hydrogen, C₁-C₆-alkyl which is unsubstituted or substituted one or more times by R¹⁰, C₂-C₆-alkenyl which is unsubstituted or substituted one or more times by R¹⁰, C₂-C₆-alkynyl which is unsubstituted or substituted one or more times by R¹⁰, C₃-C₆-cycloalkyl which is unsubstituted or substituted one or more times by R¹¹, C₃-C₈-cycloalkenyl which is unsubstituted or substituted one or more times by R¹¹, halo, OR³, SR³, NR³R⁴, aryl which is unsubstituted or substituted one or more times by R¹², heterocyclyl which is unsubstituted or substituted one or more times by R¹², S(O)ₚR¹³, S(O)ₚNR³R⁴, —C(O)R³, —C(O)OR³, —C(O)NR³R⁴, —NO₂, or CN;
R⁸ is, in each case independently,
H,
C₁-C₆-alkyl which is unsubstituted or substituted one or more times by C₁-C₆-alkoxy, C₁-C₆-haloalkoxy, OH, halo or NR³R⁴,
O—C₁-C₆-alkyl,
OH,
halo, or
NR³R⁴,
or two R⁸ together may represent oxo;
R⁹ is, in each case independently, hydrogen, C₁-C₆-alkyl which is unsubstituted or substituted one or more times by R¹⁰, C₂-C₆-alkenyl which is unsubstituted or substituted one or more times by R¹⁰, C₂-C₆-alkynyl which is unsubstituted or substituted one or more times by R¹⁰, C₃-C₆-cycloalkyl which is unsubstituted or substituted one or more times by R¹¹, C₃-C₈-cycloalkeny which is unsubstituted or substituted one or more times by R¹¹I, aryl which is unsubstituted or substituted one or more times by R¹², or heterocyclyl which is unsubstituted or substituted one or more times by R¹²;
R¹⁰ is, in each case independently, halogen, C₂-C₇-alkoxycarbonyl, hydroxy, C₁-C₆-alkoxy, C₁-C₆-haloalkoxy, C₃-C₆-alkenyloxy, C₃-C₆-alkynyloxy, nitro, amino, C₁₋₆-alkylamino, dialkylamino wherein each alkyl group has independently 1 to 6 carbon atoms, aminocarbonyl, C₁₋₆-alkyl-aminocarbonyl, dialkylaminocarbonyl wherein each alkyl group has independently 1 to 6 carbon atoms, hydroxyalkyl having 1 to 6 carbon atoms, hydroxyalkoxy having 1 to 6 carbon atoms, carboxy, cyano, formyl, alkanoyl having 2 to 7 carbon atoms, benzoyl, C₁-C₆-alkylthio, C₁-C₆-alkylsulfinyl, C₁-C₆-alkylsulfonyl, C₁-C₆-alkylsulfamoyl, phenoxy, formyloxy, alkanoyloxy having 2 to 7 carbon atoms, and benzoyloxy;
R¹¹ is, in each case independently, halogen, C₁-C₆-alkyl, halogenated C₁-C₆-alkyl, C₃-C₆-alkenyl, C₃-C₆-alkynyl, C₂-C₇-alkoxycarbonyl, hydroxy, C₁-C₆-alkoxy, C₁-C₆-haloalkoxy, C₃-C₆-alkenyloxy, C₃-C₆-alkynyloxy, nitro, methylenedioxy, ethylenedioxy, amino, C₁₋₆-alkylamino, dialkylamino wherein each alkyl group has independently 1 to 6 carbon atoms, aminocarbonyl, C₁₋₆-alkyl-aminocarbonyl, dialkylaminocarbonyl wherein each alkyl group has independently 1 to 6 carbon atoms, hydroxyalkyl having 1 to 6 carbon atoms, hydroxyalkoxy having 1 to 6 carbon atoms, carboxy, cyano, formyl, alkanoyl having 2 to 7 carbon atoms, benzoyl, C₁-C₆-alkylthio, C₁-C₆-alkylsulfinyl, C₁-C₆-alkylsulfonyl, C₁-C₆-alkylsulfamoyl, phenoxy, formyloxy, alkanoyloxy having 2 to 7 carbon atoms, and benzoyloxy;
R¹² is, in each case independently, halogen, C₁-C₆-alkyl, halogenated C₁-C₆-alkyl, C₃-C₆-alkenyl, C₃-C₆-alkynyl, C₃-C₈-cycloalkyl, C₅-C₈-cycloalkenyl, C₂-C₇-alkoxycarbonyl, hydroxy, C₁-C₆-alkoxy, C₁-C₆-haloalkoxy, C₃-C₆-alkenyloxy, C₃-C₆-alkynyloxy, nitro, methylenedioxy, ethylenedioxy, amino, C₁₋₆-alkylamino, dialkylamino wherein each alkyl group has independently 1 to 6 carbon atoms, aminocarbonyl, C₁₋₆-alkyl-aminocarbonyl, dialkylaminocarbonyl wherein each alkyl group has independently 1 to 6 carbon atoms, hydroxyalkyl having 1 to 6 carbon atoms, hydroxyalkoxy having 1 to 6 carbon atoms, carboxy, cyano, formyl, alkanoyl having 2 to 7 carbon atoms, benzoyl, C₁-C₆-alkylthio, C₁-C₆-alkylsulfinyl, C₁-C₆-alkylsulfonyl, C₁-C₆-alkylsulfamoyl, phenoxy, formyloxy, alkanoyloxy having 2 to 7 carbon atoms, and benzoyloxy; and
R¹³ is in each case independently, C₁-C₆-alkyl which is unsubstituted or substituted one or more times by R¹⁰, C₃-C₆-alkenyl which is unsubstituted or substituted one or more times by R¹⁰, C₃-C₆-alkynyl which is unsubstituted or substituted one or more times by R¹⁰, C₃-C₆-cycloalkyl which is unsubstituted or substituted one or more times by R¹¹, C₃-C₈-cycloalkenyl which is unsubstituted or substituted one or more times by R¹¹, aryl which is unsubstituted or substituted one or more times by R¹², heterocyclyl which is unsubstituted or substituted one or more times by R¹², —C(O)R⁵, —C(O)OR⁵, or —C(O)NR⁵R⁶;
or a tautomer thereof, or a pharmaceutically acceptable salt, ester, or salt of an ester thereof, and wherein if the compound exhibits chirality it can be in the form of a mixture of enantiomers or a mixture of diastereomers, or can be in the form of a single enantiomer or a single diastereomer.

2. A compound according to claim 1, wherein:
R¹ and R² are each, independently, hydrogen, C₁-C₆-alkyl which is unsubstituted or substituted one or more times by R¹⁰, C₂-C₆-alkenyl which is unsubstituted or substituted one or more times by R¹⁰, C₂-C₆-alkynyl which is unsubstituted or substituted one or more times by R¹⁰, C₃-C₆-cycloalkyl which is unsubstituted or substituted one or more times by R¹⁰, C₃-C₈-cycloalkenyl which is unsubstituted or substituted one or more times by R¹⁰, halo, OR³, SR³, NR³R⁴, aryl which is unsubstituted or substituted one or more times by R¹¹, heterocyclyl which is unsubstituted or substituted one or more times by R¹¹, S(O)ₚR¹³, S(O)ₚNR³R⁴, —C(O)R³, —C(O)OR³, —C(O)NR³R⁴, NO₂, or CN, or
R¹ and R² taken together, are 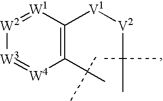
or —(CH₂)ₘ—.

3. A compound according to claim 1, wherein:
X is NH, N(CH₃), S or O;
R¹ and R² are each, independently, hydrogen, C₁-C₆-alkyl which is unsubstituted or substituted one or more times by R¹⁰, C₃-C₆-cycloalkyl which is unsubstituted or substituted one or more times by R¹⁰, halo, NH₂, phenyl which is unsubstituted or substituted one or more times by R¹¹, naphthyl which is unsubstituted or substituted one or more times by R¹¹, 1,4-benzodioxan-6-yl which is unsubstituted or substituted one or more times by R¹¹, pyridyl which is unsubstituted or substituted one or more times by R¹¹, thienyl which is unsubstituted or substituted one or more times by R¹¹, or NO₂, or
R¹ and R² taken together, are 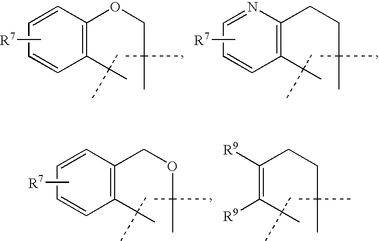
or, —(CH₂)ₘ; and
m is 3, 4, 5, or 6.

4. A compound according to claim 1, wherein:
R¹ and R² are each, independently, hydrogen, C₁-C₆-alkyl which is unsubstituted or substituted one or more times by R¹⁰, C₃-C₆-cycloalkyl which is unsubstituted or substituted one or more times by R¹⁰, halo, NH₂, phenyl which is unsubstituted or substituted one or more times by R¹¹, naphthyl which is unsubstituted or substituted one or more times by R¹¹, 1,4-benzodioxan-6-yl which is unsubstituted or substituted one or more times by R¹¹, pyridyl which is unsubstituted or substituted one or more times by R¹¹, thienyl which is unsubstituted or substituted one or more times by R¹¹, or NO₂, or
R¹ and R² taken together, are 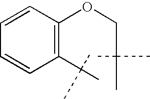
or, —(CH₂)ₘ.

5. A compound according to claim 1, wherein R¹ and R² are each independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, carbamoyl (NH₂—CO—), phenyl, methylphenyl, ethylphenyl, methoxyphenyl, ethoxyphenyl, fluorophenyl, chlorophenyl, aminophenyl, cyanophenyl, nitrophenyl, naphthyl, pyridyl, methylpyridyl, ethylpyridyl, methoxypyridyl, ethoxypyridyl, fluoropyridyl, chloropyridyl, aminopyridyl, cyanopyridyl, nitropyridyl, thienyl, methylthienyl, fluorothienyl, chlorothienyl, pyazole, or methylpyrazole.

6. A compound according to claim 1, wherein
R¹ is H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—), and/or
R² is H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, carbamoyl (NH₂—CO—), phenyl, methylphenyl, ethylphenyl, methoxyphenyl, ethoxyphenyl, fluorophenyl, chlorophenyl, aminophenyl, cyanophenyl, nitrophenyl, naphthyl, pyridyl, methylpyridyl, ethylpyridyl, methoxypyridyl, ethoxypyridyl, fluoropyridyl, chloropyridyl, aminopyridyl, cyanopyridyl, nitropyridyl, thienyl, methylthienyl, fluorothienyl, chlorothienyl, pyazole, or methylpyrazole.

7. A compound according to claim 1, wherein
R¹ is H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—), and/or
R² is phenyl, methylphenyl, ethylphenyl, methoxyphenyl, ethoxyphenyl, fluorophenyl, chlorophenyl, aminophenyl, cyanophenyl, nitrophenyl, naphthyl, pyridyl, methylpyridyl, ethylpyridyl, methoxypyridyl, ethoxypyridyl, fluoropyridyl, chloropyridyl, aminopyridyl, cyanopyridyl, nitropyridyl, thienyl, methylthienyl, fluorothienyl, chlorothienyl, pyazole, or methylpyrazole.

8. A compound according to claim 1, wherein R¹ and R² together are —(CH₂)₃—, —(CH₂)₄—, —(CH₂)₅—, or —(CH₂)₆—.

9. A compound according to claim 8, wherein R¹ and R² together are —(CH₂)₃— or —(CH₂)₆—.

10. A compound according to claim 1, wherein R¹ and R² together are

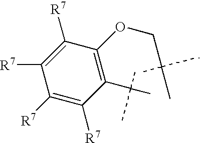

11. A compound according to claim 1, wherein X is NH, S or O.

12. A compound according to claim 11, wherein X is O.

13. A compound according to claim 1, wherein said compound is of Formula Ia:

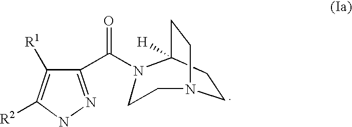

14. A compound according to claim 13, wherein R¹ and R² are each independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, carbamoyl (NH₂—CO—), phenyl, methylphenyl, ethylphenyl, methoxyphenyl, ethoxyphenyl, fluorophenyl, chlorophenyl, aminophenyl, cyanophenyl, nitrophenyl, naphthyl, pyridyl, methylpyridyl, ethylpyridyl, methoxypyridyl, ethoxypyridyl, fluoropyridyl, chloropyridyl, aminopyridyl, cyanopyridyl, nitropyridyl, thienyl, methylthienyl, fluorothienyl, chlorothienyl, pyazole, or methylpyrazole.

15. A compound according to claim 13, wherein
R¹ is H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—), and/or
R² is H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, carbamoyl (NH₂—CO—), phenyl, methylphenyl, ethylphenyl, methoxyphenyl, ethoxyphenyl, fluorophenyl, chlorophenyl, aminophenyl, cyanophenyl, nitrophenyl, naphthyl, pyridyl, methylpyridyl, ethylpyridyl, methoxypyridyl, ethoxypyridyl, fluoropyridyl, chloropyridyl, aminopyridyl, cyanopyridyl, nitropyridyl, thienyl, methylthienyl, fluorothienyl, chlorothienyl, pyazole, or methylpyrazole.

16. A compound according to claim 13, wherein
R¹ is H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, and carbamoyl (NH₂—CO—), and/or
R² is phenyl, methylphenyl, ethylphenyl, methoxyphenyl, ethoxyphenyl, fluorophenyl, chlorophenyl, aminophenyl, cyanophenyl, nitrophenyl, naphthyl, pyridyl, methylpyridyl, ethylpyridyl, methoxypyridyl, ethoxypyridyl, fluoropyridyl, chloropyridyl, aminopyridyl, cyanopyridyl, nitropyridyl, thienyl, methylthienyl, fluorothienyl, chlorothienyl, pyazole, or methylpyrazole.

17. A compound according to claim 13, wherein R¹ and R² together are —(CH₂)₃—, —(CH₂)₄—, —(CH₂)₅—, or —(CH₂)₆—.

18. A compound according to claim 17, wherein R¹ and R² together are —(CH₂)₃— or —(CH₂)₆—.

19. A compound according to claim 1, wherein said compound is of Formula Ib:

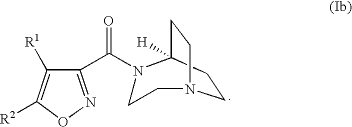

20. A compound according to claim 19, wherein R¹ and R² are each independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, carbamoyl (NH₂—CO—), phenyl, methylphenyl, ethylphenyl, methoxyphenyl, ethoxyphenyl, fluorophenyl, chlorophenyl, aminophenyl, cyanophenyl, or nitrophenyl.

21. A compound according to claim 1, wherein said compound is of Formula Ic:

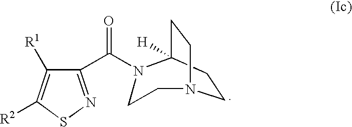

22. A compound according to claim 21, wherein R¹ and R² are each independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, carbamoyl (NH₂—CO—), phenyl, methylphenyl, ethylphenyl, methoxyphenyl, ethoxyphenyl, fluorophenyl, chlorophenyl, aminophenyl, cyanophenyl, or nitrophenyl.

23. A compound according to claim 1, wherein said compound is of Formula Id:

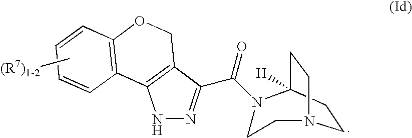

24. A compound according to claim 23, wherein R⁷ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, carbamoyl (NH₂—CO—), or methoxypyrrolidinyl.

25. A compound according to claim 23, wherein there is only one R⁷ group and said one R⁷ group is H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

26. A compound according to claim 23, wherein R⁷ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

27. A compound according to claim 1, wherein said compound is of Formula Ie:

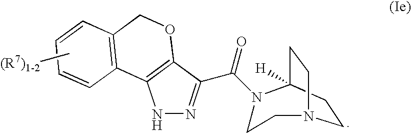

28. A compound according to claim 27, wherein R⁷ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

29. A compound according to claim 27, wherein R⁷ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

30. A compound according to claim 1, wherein said compound is of Formula If:

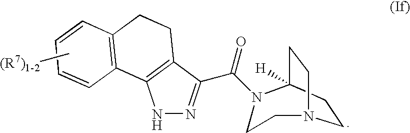

31. A compound according to claim 30, wherein R⁷ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

35. A compound according to claim 30, wherein R⁷ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

36. A compound according to claim 1, wherein said compound is of Formula Ig:

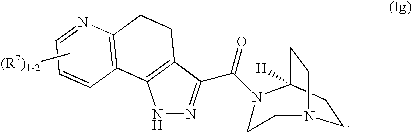

37. A compound according to claim 36, wherein R⁷ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

38. A compound according to claim 37, wherein R⁷ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

39. A compound according to claim 1, wherein said compound is of Formula Ih:

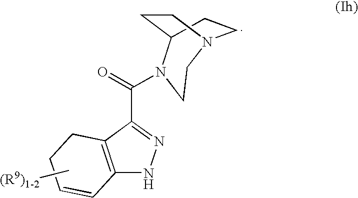

40. A compound according to claim 39, wherein R⁹ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

41. A compound according to claim 39, wherein R⁹ is in each case independently H, F, Cl, Br, cyano, methyl, ethyl, propyl, hydroxy, methoxy, ethoxy, nitro, amino, methylamino, ethylamino, dimethylamino, diethylamino, cyclopropyl, cyclopentyl, cyclohexyl, acetyl, propionyl, acetoxy, methoxycarbonyl, ethoxycarbonyl, or carbamoyl (NH₂—CO—).

42. A compound according to claim 1, wherein said compound is selected from:
4-[(4-Chloro-1H-pyrazol-3-yl)carbonyl]-1,4-diazabicyclo[3.2.2]nonane,
4-[(4-nitro-1H-pyrazol-3-yl)carbonyl]-1,4-diazabicyclo[3.2.2]nonane,
4-[(5-Methyl-1H-pyrazol-3-yl)carbonyl]-1,4-diazabicyclo[3.2.2]nonane,
4-(1H-pyrazol-3-ylcarbonyl)-1,4-diazabicyclo[3.2.2]nonane,
4-{[5-(4-Methoxyphenyl)-1H-pyrazol-3-yl]carbonyl}-1,4-diazabicyclo[3.2.2]nonane,
4-[(5-Phenylisoxazol-3-yl)carbonyl]-1,4-diazabicyclo[3.2.2]nonane,
4-{[5-(2-thienyl)-1H-pyrazol-3-yl]carbonyl}-1,4-diazabicyclo[3.2.2]nonane,
4-[(5-Phenyl-1H-pyrazol-3-yl)carbonyl]-1,4-diazabicyclo[3.2.2]nonane,
4-[(5-Cyclopropyl-1H-pyrazol-3-yl)carbonyl]-1,4-diazabicyclo[3.2.2]nonane,
4-{[5-(3-Methoxyphenyl)-1H-pyrazol-3-yl]carbonyl}-1,4-diazabicyclo[3.2.2]nonane,
4-(1,4,5,6-Tetrahydrocyclopenta[c]pyrazol-3-ylcarbonyl)-1,4-diazabicyclo[3.2.2]nonane,
4-{[5-(2-Methoxyphenyl)-1H-pyrazol-3-yl]carbonyl}-1,4-diazabicyclo[3.2.2]nonane,
4-[(4-Bromo-1H-pyrazol-3-yl)carbonyl]-1,4-diazabicyclo[3.2.2]nonane,
3-(1,4-Diazabicyclo[3.2.2]non-4-ylcarbonyl)isothiazol-4-amine,
3-(1,4-Diazabicyclo[3.2.2]non-4-ylcarbonyl)-1,4-dihydrochromeno[4,3-c]pyrazole,
3-(1,4-Diazabicyclo[3.2.2]non-4-ylcarbonyl)-4,5,6,7,8,9-hexahydro-1H-cycloocta[c]pyrazole,
4-{[5-(2,3-Dihydro-1,4-benzodioxin-6-yl)-1H-pyrazol-3-yl]carbonyl}-1,4-diazabicyclo[3.2.2]nonane,
4-{[5-(2-Naphthyl)-1H-pyrazol-3-yl]carbonyl}-1,4-diazabicyclo[3.2.2]nonane,
4-{[5-(3-Thienyl)-1H-pyrazol-3-yl]carbonyl}-1,4-diazabicyclo[3.2.2]nonane,
4-{[5-(4-Fluorophenyl)-1H-pyrazol-3-yl]carbonyl}-1,4-diazabicyclo[3.2.2]nonane,
4-[(5-Pyridin-2-yl-1H-pyrazol-3-yl)carbonyl]-1,4-diazabicyclo[3.2.2]nonane,
4-[(5-Pyridin-4-yl-1H-pyrazol-3-yl)carbonyl]-1,4-diazabicyclo[3.2.2]nonane,
4-[(5-Pyridin-3-yl-1H-pyrazol-3-yl)carbonyl]-1,4-diazabicyclo[3.2.2]nonane,
5′-(1,4-Diazabicyclo[3.2.2]non-4-ylcarbonyl)-1-methyl-1H,2′H-3,3′-bipyrazole,
3-(1,4-Diazabicyclo[3.2.2]non-4-ylcarbonyl)-6-methoxy-1,4-dihydrochromeno[4,3-c]pyrazole,
3-(1,4-Diazabicyclo[3.2.2]non-4-ylcarbonyl)-8-methoxy-1,4-dihydrochromeno[4,3-c]pyrazole,
3-(1,4-Diazabicyclo[3.2.2]non-4-ylcarbonyl)-7-methoxy-1,4-dihydrochromeno[4,3-c]pyrazole,
3-(1,4-Diazabicyclo[3.2.2]non-4-ylcarbonyl)-6-(4-fluorophenyl)-4,5-dihydro-1H-indazole
4-(Isothiazol-3-ylcarbonyl)-1,4-diazabicyclo[3.2.2]nonane
4-[(5-Bromoisothiazol-3-yl)carbonyl]-1,4-diazabicyclo[3.2.2]nonane
3-(1,4-Diazabicyclo[3.2.2]non-4-ylcarbonyl)-4,5-dihydro-1H-benzo[g]indazole
3-(1,4-Diazabicyclo[3.2.2]non-4-ylcarbonyl)-1,5-dihydroisochromeno[4,3-c]pyrazole
3-(1,4-Diazabicyclo[3.2.2]non-4-ylcarbonyl)-4,5-dihydro-1H-pyrazolo[3,4-f]quinoline
3-(1,4-Diazabicyclo[3.2.2]non-4-ylcarbonyl)-7-[(3S)-3-methoxypyrrolidin-1-yl]-1,4-dihydrochromeno[4,3-c]pyrazole
7-Bromo-3-(1,4-diazabicyclo[3.2.2]non-4-ylcarbonyl)-1,4-dihydrochromeno[4,3-c]pyrazole,
3-(1,4-Dzabicyclo[3.2.2]non-4-ylcarbonyl)-1H-pyrazol-5-amine, and
tautomers thereof, and pharmaceutically acceptable salts thereof, and
wherein if the compound exhibits chirality said compound can be in the form of a mixture of enantiomers or a mixture of diastereomers, or said compound can be in the form of a single enantiomer or a single diastereomer.

43. A compound according to claim 42, wherein said compound is selected from:
4-[(4-Chloro-1H-pyrazol-3-yl)carbonyl]-1,4-diazabicyclo[3.2.2]nonane,
4-[(4-nitro-1H-pyrazol-3-yl)carbonyl]-1,4-diazabicyclo[3.2.2]nonane,
4-[(5-Methyl-1H-pyrazol-3-yl)carbonyl]-1,4-diazabicyclo[3.2.2]nonane,
4-(1H-pyrazol-3-ylcarbonyl)-1,4-diazabicyclo[3.2.2]nonane,
4-{[5-(4-Methoxyphenyl)-1H-pyrazol-3-yl]carbonyl}-1,4-diazabicyclo[3.2.2]nonane,
4-[(5-Phenylisoxazol-3-yl)carbonyl]-1,4-diazabicyclo[3.2.2]nonane,
4-{[5-(2-thienyl)-1H-pyrazol-3-yl]carbonyl}-1,4-diazabicyclo[3.2.2]nonane,
4-[(5-Phenyl-1H-pyrazol-3-yl)carbonyl]-1,4-diazabicyclo[3.2.2]nonane,
4-[(5-Cyclopropyl-1H-pyrazol-3-yl)carbonyl]-1,4-diazabicyclo[3.2.2]nonane,
4-{[5-(3-Methoxyphenyl)-1H-pyrazol-3-yl]carbonyl}-1,4-diazabicyclo[3.2.2]nonane,
4-(1,4,5,6-Tetrahydrocyclopenta[c]pyrazol-3-ylcarbonyl)-1,4-diazabicyclo[3.2.2]nonane,
4-{[5-(2-Methoxyphenyl)-1H-pyrazol-3-yl]carbonyl}-1,4-diazabicyclo[3.2.2]nonane,
4-[(4-Bromo-1H-pyrazol-3-yl)carbonyl]-1,4-diazabicyclo[3.2.2]nonane,
3-(1,4-Diazabicyclo[3.2.2]non-4-ylcarbonyl)isothiazol-4-amine,
3-(1,4-Diazabicyclo[3.2.2]non-4-ylcarbonyl)-1,4-dihydrochromeno[4,3-c]pyrazole,
3-(1,4-Diazabicyclo[3.2.2]non-4-ylcarbonyl)-4,5,6,7,8,9-hexahydro-1H-cycloocta[c]pyrazole,
4-{[5-(2,3-Dihydro-1,4-benzodioxin-6-yl)-1H-pyrazol-3-yl]carbonyl}-1,4-diazabicyclo[3.2.2]nonane,
4-{[5-(2-Naphthyl)-1H-pyrazol-3-yl]carbonyl}-1,4-diazabicyclo[3.2.2]nonane,
4-{[5-(3-Thienyl)-1H-pyrazol-3-yl]carbonyl}-1,4-diazabicyclo[3.2.2]nonane,
4-{[5-(4-Fluorophenyl)-1H-pyrazol-3-yl]carbonyl}-1,4-diazabicyclo[3.2.2]nonane,
4-[(5-Pyridin-2-yl-1H-pyrazol-3-yl)carbonyl]-1,4-diazabicyclo[3.2.2]nonane,
4-[(5-Pyridin-4-yl-1H-pyrazol-3-yl)carbonyl]-1,4-diazabicyclo[3.2.2]nonane,
4-[(5-Pyridin-3-yl-1H-pyrazol-3-yl)carbonyl]-1,4-diazabicyclo[3.2.2]nonane,
5′-(1,4-Diazabicyclo[3.2.2]non-4-ylcarbonyl)-1-methyl-1H,2′H-3,3′-bipyrazole,
3-(1,4-Diazabicyclo[3.2.2]non-4-ylcarbonyl)-6-methoxy-1,4-dihydrochromeno[4,3-c]pyrazole,
3-(1,4-Diazabicyclo[3.2.2]non-4-ylcarbonyl)-8-methoxy-1,4-dihydrochromeno[4,3-c]pyrazole,
3-(1,4-Diazabicyclo[3.2.2]non-4-ylcarbonyl)-7-methoxy-1,4-dihydrochromeno[4,3-c]pyrazole,
3-(1,4-Diazabicyclo[3.2.2]non-4-ylcarbonyl)-6-(4-fluorophenyl)-4,5-dihydro-1H-indazole
4-(Isothiazol-3-ylcarbonyl)-1,4-diazabicyclo[3.2.2]nonane
4-[(5-Bromoisothiazol-3-yl)carbonyl]-1,4-diazabicyclo[3.2.2]nonane
3-(1,4-Diazabicyclo[3.2.2]non-4-ylcarbonyl)-4,5-dihydro-1H-benzo[g]indazole
3-(1,4-Diazabicyclo[3.2.2]non-4-ylcarbonyl)-1,5-dihydroisochromeno[4,3-c]pyrazole
3-(1,4-Diazabicyclo[3.2.2]non-4-ylcarbonyl)-4,5-dihydro-1H-pyrazolo[3,4-f]quinoline
3-(1,4-Diazabicyclo[3.2.2]non-4-ylcarbonyl)-7-[(3S)-3-methoxypyrrolidin-1-yl]-1,4-dihydrochromeno[4,3-c]pyrazole
7-Bromo-3-(1,4-diazabicyclo[3.2.2]non-4-ylcarbonyl)-1,4-dihydrochromeno[4,3-c]pyrazole, and
tautomers thereof, and pharmaceutically acceptable salts thereof, and
wherein if the compound exhibits chirality said compound can be in the form of a mixture of enantiomers or a mixture of diastereomers, or said compound can be in the form of a single enantiomer or a single diastereomer.

44. A compound according to claim 42, wherein said compound is selected from:
4-[(4-Chloro-1H-pyrazol-3-yl)carbonyl]-1,4-diazabicyclo[3.2.2]nonane,
4-[(4-nitro-1H-pyrazol-3-yl)carbonyl]-1,4-diazabicyclo[3.2.2]nonane,
4-[(5-Methyl-1H-pyrazol-3-yl)carbonyl]-1,4-diazabicyclo[3.2.2]nonane,
4-(1H-pyrazol-3-ylcarbonyl)-1,4-diazabicyclo[3.2.2]nonane,
4-{[5-(4-Methoxyphenyl)-1H-pyrazol-3-yl]carbonyl}-1,4-diazabicyclo[3.2.2]nonane,
4-[(5-Phenylisoxazol-3-yl)carbonyl]-1,4-diazabicyclo[3.2.2]nonane,
4-{[5-(2-thienyl)-1H-pyrazol-3-yl]carbonyl}-1,4-diazabicyclo[3.2.2]nonane,
4-[(5-Phenyl-1H-pyrazol-3-yl)carbonyl]-1,4-diazabicyclo[3.2.2]nonane,
4-[(5-Cyclopropyl-1H-pyrazol-3-yl)carbonyl]-1,4-diazabicyclo[3.2.2]nonane,
4-{[5-(3-Methoxyphenyl)-1H-pyrazol-3-yl]carbonyl}-1,4-diazabicyclo[3.2.2]nonane,
4-(1,4,5,6-Tetrahydrocyclopenta[c]pyrazol-3-ylcarbonyl)-1,4-diazabicyclo[3.2.2]nonane,
4-{[5-(2-Methoxyphenyl)-1H-pyrazol-3-yl]carbonyl}-1,4-diazabicyclo[3.2.2]nonane,
4-[(4-Bromo-1H-pyrazol-3-yl)carbonyl]-1,4-diazabicyclo[3.2.2]nonane,
3-(1,4-Diazabicyclo[3.2.2]non-4-ylcarbonyl)isothiazol-4-amine,
3-(1,4-Diazabicyclo[3.2.2]non-4-ylcarbonyl)-1,4-dihydrochromeno[4,3-c]pyrazole,
3-(1,4-Diazabicyclo[3.2.2]non-4-ylcarbonyl)-4,5,6,7,8,9-hexahydro-1H-cycloocta[c]pyrazole,
4-{[5-(2,3-Dihydro-1,4-benzodioxin-6-yl)-1H-pyrazol-3-yl]carbonyl}-1,4-diazabicyclo[3.2.2]nonane,
4-{[5-(2-Naphthyl)-1H-pyrazol-3-yl]carbonyl}-1,4-diazabicyclo[3.2.2]nonane,
4-{[5-(3-Thienyl)-1H-pyrazol-3-yl]carbonyl}-1,4-diazabicyclo[3.2.2]nonane,
4-{[5-(4-Fluorophenyl)-1H-pyrazol-3-yl]carbonyl}-1,4-diazabicyclo[3.2.2]nonane,
4-[(5-Pyridin-2-yl-1H-pyrazol-3-yl)carbonyl]-1,4-diazabicyclo[3.2.2]nonane,
4-[(5-Pyridin-4-yl-1H-pyrazol-3-yl)carbonyl]-1,4-diazabicyclo[3.2.2]nonane,
4-[(5-Pyridin-3-yl-1H-pyrazol-3-yl)carbonyl]-1,4-diazabicyclo[3.2.2]nonane,
5′-(1,4-Diazabicyclo[3.2.2]non-4-ylcarbonyl)-1-methyl-1H,2′H-3,3′-bipyrazole,
3-(1,4-Diazabicyclo[3.2.2]non-4-ylcarbonyl)-6-methoxy-1,4-dihydrochromeno[4,3-c]pyrazole,
3-(1,4-Diazabicyclo[3.2.2]non-4-ylcarbonyl)-8-methoxy-1,4-dihydrochromeno[4,3-c]pyrazole,
3-(1,4-Diazabicyclo[3.2.2]non-4-ylcarbonyl)-7-methoxy-1,4-dihydrochromeno[4,3-c]pyrazole, and
tautomers thereof, and pharmaceutically acceptable salts thereof, and
wherein if the compound exhibits chirality said compound can be in the form of a mixture of enantiomers or a mixture of diastereomers, or said compound can be in the form of a single enantiomer or a single diastereomer.

45. A compound according to claim 42, wherein said compound is selected from:
(1H-Pyrazol-3-ylcarbonyl)-1,4-diazabicyclo[3.2.2]nonane hydrochloride, and
3-(1,4-Diazabicyclo[3.2.2]non-4-ylcarbonyl)-1,4-dihydrochromeno[4,3-c]pyrazole hydrochloride,
and
tautomers thereof, and pharmaceutically acceptable salts thereof, and
wherein if the compound exhibits chirality said compound can be in the form of a mixture of enantiomers or a mixture of diastereomers, or said compound can be in the form of a single enantiomer or a single diastereomer.

46. A pharmaceutical composition comprising a compound according to any one of claims 1 to 45, and a pharmaceutically acceptable carrier.

47. A pharmaceutical composition according to claim 46, further comprising at least one additional active agent, wherein said additional active agent is another a-7 agonist, a PDE4 inhibitor, a calcium channel blocker, a muscarinic m1 or m2 modulator, an adenosine receptor modulator, an ampakine, an NMDA-R modulator, an mGluR modulator, a dopamine modulator, a serotonin modulator, a cannabinoid modulator, a cholinesterase inhibitors, an agent for treatment of ADHD, an anti-depressant, anti-inflammatory agent, ananti-psychotic agent, a beta secretase modulator, a bipolar disorder agent, a GABA-nergic drug, a gamma secretase modulator, a histamine H3, a kinase inhibitor, a MAO-B inhibitor, a mood stabilizer, a 5HT4 modulating agent, a 5HT6 antagonist, or an α4β2 modulating agent.

48. A method of treating a patient suffering from a psychotic disease, a neurodegenerative disease involving a dysfunction of the cholinergic system, and/or a condition of memory and/or cognition impairment, comprising administering to the patient an effective amount of a compound according to any one of claims 1 to 45.

49. A method according to claim 48, wherein said patient is suffering from schizophrenia, anxiety, mania, depression, or manic depression.

50. A method according to claim 48, wherein said patient is suffering from Tourette's syndrome, Parkinson's disease, or Huntington's .

51. A method according to claim 48, wherein said patient is suffering from Alzheimer's disease, Lewy Body Dementia, Amyotrophic Lateral Sclerosis, memory impairment, memory loss, cognition deficit, attention deficit, or Attention Deficit Hyperactivity Disorder.

52. A method according to claim 48, wherein said patient is suffering from Alzheimer's diseases, Pick's disease, diffuse Lewy Body disease, progressive supranuclear palsy (Steel-Richardson syndrome), multisystem degeneration (Shy-Drager syndrome), motor neuron diseases including amyotrophic lateral sclerosis (ALS), degenerative ataxias, cortical basal degeneration, ALS-Parkinson's-Dementia complex of Guam, subacute sclerosing panencephalitis, Huntington's disease, Parkinson's disease, synucleinopathies, primary progressive aphasia, striatonigral degeneration, Machado-Joseph disease/spinocerebellar ataxia type 3, olivopontocerebellar degenerations, Gilles De La Tourette's disease, bulbar palsy, pseudobulbar palsy, spinal muscular atrophy, spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leu koencephalopathy, Creutzfeldt-Jakob disease, Gerstmann-Sträussler-Scheinker disease, Kuru, or fatal familial insomnia.

53. A method according to claim 48, wherein said patient is suffering froma neurodegenerative disorder resulting from cerebral ischemia, infarction, intracranial hemorrhage, or intracranial and intravertebral lesions.

54. A method according to claim 48, wherein said patient is suffering from age-related dementia or other dementia, or conditions with memory loss.

55. A method according to claim 48, wherein said patient is suffering from memory impairment due to Alzheimer's disease, mild cognitive impairment due to aging, schizophrenia, Parkinson's disease, Huntington's disease, Pick's disease, Creutzfeldt-Jakob disease, depression, aging, head trauma, stroke, CNS hypoxia, cerebral senility, multiinfarct dementia, HIV, or cardiovascular disease.

56. A method according to claim 48, wherein said patient is suffering from memory impairment as a result of chemotherapy, kidney dialysis, post-operative surgery, or a bipolar disorder.

57. A method according to claim 48, wherein said patient is suffering from dementia due to Alzheimer's disease.

58. A method according to claim 48, wherein said patient is suffering from hereditary cerebral angiopathy, nonneuropathic hereditary amyloid, Down's syndrome, macroglobulinemia, secondary familial Mediterranean fever, Muckle-Wells syndrome, multiple myeloma, pancreatic- or cardiac-related amyloidosis, chronic hemodialysis anthropathy, Finnish amyloidosis , or Iowa amyloidosis.

59. A method of treating a patient for alcohol withdrawal or treating a patient with anti-intoxication therapy comprising administering to the patient an effective amount of a compound according to any one of claims 1 to 45.

60. A method of treating a patient suffering from nicotine addiction, pain, jetlag, obesity and/or diabetes, or a method of inducing smoking cessation in a patient comprising administering to the patient an effective amount of a compound according to any one of claims 1 to 45.

60. A method of treating a patient suffering from inflammation comprising administering to the patient an effective amount of a compound according to any one of claims 1 to 45.

61. A method according to claim 60, wherein said inflammation is due to rheumatoid arthritis, diabetes, sepsis, an autoimmune disease, fibromyalgia, or ulcerative colitis.
